(19) United States
(12) Patent Application Publication
(10) Pub. No.: US 2005/0221337 A1
(43) Pub. Date: Oct. 6, 2005

(54) Microarrays and microspheres comprising oligosaccharides, complex carbohydrates or glycoproteins

(75) Inventors: Peter H. Seeberger, Zurich (CH); Daniel M. Ratner, Cambridge, MA (US); Eddie W. Adams, Brighton, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Massachusetts Institute of Technology, Cambridge, MA (US)

(21) Appl. No.: 10/950,701

(22) Filed: Sep. 27, 2004

Related U.S. Application Data
(60) Provisional application No. 60/508,209, filed on Oct. 2, 2003.

(51) Int. Cl. C12Q 1/68; C12N 9/10; C12M 1/34

(52) U.S. Cl. 435/6; 435/287.2

(57) ABSTRACT

One aspect of the present invention relates to an array, comprising a plurality of spots on a solid support, wherein each spot independently comprises a substrate attached to said solid support, wherein each substrate attached to said solid support is independently a carbohydrate-containing molecule. A second aspect of the present invention relates to a method of preparing such an array of carbohydrate-containing molecules. A third aspect of the present invention relates to a method to detect the interaction of a carbohydrate with a binding molecule.

### RELATED APPLICATIONS

This application claims the benefit of priority to U.S. Provisional Patent Application Ser. No. 60/508,209, filed Oct. 2, 2003; the entirety of which is hereby incorporated by reference.

### BACKGROUND OF THE INVENTION

Carbohydrates are known to play a key role in numerous biological processes, such as immune response, viral membrane fusion, and glycoprotein homeostasis. Research into the biochemical role of carbohydrates has revealed that in many cases a carbohydrate molecule is bonded to another biomolecule to form a glycoconjugate (e.g. glycopeptides, glycolipids, glycosaminoglycans and proteoglyans). In fact, glycoconjugates have been linked to processes controlling inflammation, cell-cell interactions, signal transduction, fertility and development. See G. Kansas Blood 1996, 88, 3259; J. C. Sacchettini et al. Biochemistry 2001, 40, 3009, and V. D. Vacquier et al. Dev. Genetics 1997, 192, 125. As a result, there is substantial interest in gaining a better understanding of the physiological role of carbohydrates owing to their participation in these fundamental cellular processes.

One area of current biochemical research focuses on examining the interactions between carbohydrates and proteins because binding of carbohydrates to proteins is a key aspect of the biological function of the two classes of molecules. Hence, it is of interest to examine the factors that govern this process. However, research in this area is often hampered by several limitations. First, current substrate binding experiments often require a substantial quantity of material. This represents a limitation because many naturally-occurring compounds can only be isolated in small quantities. In addition, it is often desirable to study binding between the substrate and several hundred different receptors; such a study may not be feasible if the binding experiment requires a large quantity of material. Another limitation is the undue amount of time required to carryout a large number of individual experiments. Importantly, carbohydrate microarrays appear to address the limitations described above.

An array is an orderly arrangement of samples. Microarrays of biological materials are comprised of a number of small discrete deposits of biological materials, such as DNA, RNA, proteins, or carbohydrates, arranged in predetermined patterns on a solid support. The deposits are generally very small (e.g., in the range 100-200 μm in diameter) which allows for fabrication of plates containing a large number of deposits for conducting a large number of separate experiments. To maximize productivity, microarrays are generally prepared using robotics. The solid support can be glass, a polymer, or metal surface. Microarray technology has proven to be very effective for the study of DNA, RNA, and proteins.

Microarrays that contain carbohydrate molecules have been reported. The carbohydrate can be bound to solid support by a covalent or noncovalent interaction. The mode of attachment is limited by the necessity that the bond formed between the solid support and the carbohydrate is both durable and does not interfer with testing assays, e.g. binding affinity or biological activity. See D. Schena et al. Science 1995, 270, 467 and S. L. Schreiber et al. Science 2000, 289, 176. In one example, nitrocellulose coated slides were employed for the noncovalent immobilization of microbial polysaccharides and neoglycolipid modified oligosaccharides S. Fukui et al. Nat. Biotechnol. 2002, 20, 1011. Wong and coworkers used hydrophobic interactions to anchor lipid-bearing carbohydrates onto polystyrene microtiter plates C. H. Wong al. J. Am. Chem. Soc. 2002, 124, 14397. This technology entailed forming triazole rings by 1,3-dipolar cycloaddition of alkynes and azides wherein the azido group was attached to the carbohydrate via an ethylene tether. The triazole ring functioned as a hydrophobic anchor to a solid support comprised of saturated hydrocarbon chains 13-15 carbons in length. However, noncovalent bonds are not as strong as a covalent bonds. In one approach to covalent attachment of a carbohydrate to a solid support, Diels-Alder-mediated covalent immobilization of cyclopentadiene-derivatized monosaccharides was achieved on a gold surface bearing benzoquinone groups B. Houseman and M. Mrksich Chem. Biol. 2002, 9, 443. Another covalent immobilization technology involved reacting maleimide functionalized mono- and di-saccharide glycosylamines with a thiol-derivatized glass slide, or alternatively, thiol-functionalized carbohydrates with a self-assembled monolayer presenting maleimide groups. See S. Park et al. Angew. Chem. Int. Ed. 2002, 41, 3180.

### SUMMARY OF THE INVENTION

The invention relates generally to a method for studying the molecular binding properties of carbohydrate molecules immobilized on a solid support. One aspect of the present invention relates to an array consisting of a plurality of spots, each comprising a carbohydrate molecule attached to a solid support. The carbohydrate molecule is any monosaccharide, oligosaccharide, polysaccharide, or glycoprotein. In certain preferred embodiments, the carbohydrate is mannose, galactose, or glycoprotein gp120. The carbohydrate molecule may be attached to the solid support via a linker, e.g. bovine serum albumin. In certain preferred embodiments, the carbohydrate molecule is attached to the linker by a sulfide bond. This procedure allows carbohydrate substrates drawn from solution phase chemistry, solid phase chemistry, and/or natural sources to be readily incorporated into the present method. Furthermore, the carbohydrate can be bound to the solid support at varying concentration densities to permit determination of relative binding affinities. In other preferred embodiments, the array consists of spots that are about 120 μm in diameter and the distance between adjacent spots is about 300 μm. The high density of the present array is advantageous because it requires only a minute amount of carbohydrate substrate and is amenable to high-throughput technology.

Another aspect of the present invention relates to a method of preparing an array of carbohydrate molecules, comprising the steps of applying a carbohydrate compound to a support to form a localized spot that is about 120 μm in diameter and a distance of about 300 μm from an adjacent spot. The carbohydrate molecule is any monosaccharide, oligosaccharide, polysaccharide, or glycoprotein. In certain preferred embodiments, the carbohydrate is mannose, galactose, or glycoprotein gp120. The carbohydrate molecule may be attached to the solid support via a linker, e.g., bovine serum albumin. In certain preferred embodiments, the carbohydrate molecule is attached to the linker by a sulfide bond. In addition, the carbohydrate microarray is prepared using precision printing robotics. The rapid construction of large arrays enables many carbohydrate-binding experiments to be conducted efficiently and with little waste.

Another aspect of the present invention relates to a method to detect the interaction of a carbohydrate with a binding molecule comprising the steps of contacting a binding molecule to a carbohydrate array and detecting the presence of a complex formed between the carbohydrate and binding molecule. The binding molecule is a protein, lipid, glycoprotein, DNA, RNA, or small organic molecule that contains a detectable functional group. In preferred embodiments, the binding molecule is detected by fluorescence spectroscopy, such as in BODIPY-labeled cyanoviron-N or FITC-labeled Concanavalin A. In other embodiments, multiple proteins are tagged with different fluorescent labels for use in binding competition experiments.

Another aspect of the present invention relates to a method to determine the interaction of a carbohydrate with a molecule of interest comprising the steps of contacting a binding molecule to a carbohydrate bound to the surface of a microsphere and detecting the presence of a complex formed between the carbohydrate and binding molecule. The range of carbohydrate substrates and binding proteins discussed above for the carbohydrate microarray can be employed in the carbohydrate microsphere binding studies. In preferred embodiments, the binding event is detected by fluorescence spectroscopy. The microsphere may be composed of glass and optionally contain a signature dye to facilitate identification of the microsphere. In other preferred embodiments, the carbohydrate is bonded to the microsphere via a linker, such as bovine serum albumin wherein the carbohydrate molecule is bound to the linker by a sulfide bond.

### BRIEF DESCRIPTION OF FIGURES

FIG. 1 depicts covalent immobilization of thiol-derivatized saccharides through maleimide activated BSA-coated slides.

FIG. 2 depicts example of incorporation of thiol-containing linker.

FIG. 3 depicts proposed procedure for incorporation of thiol-containing linker into solution or solid-phase derived pentenyl-saccharides.

FIG. 4 depicts proposed procedure for incorporation of thiol-containing linker into naturally derived sugars.

FIG. 5 depicts mannose and galactose incubated with FITC-labeled ConA.

FIG. 6 depicts a monosaccharide and high-mannose oligosaccharides.

FIG. 7 depicts a high-mannose/monosaccharide array incubated with BODIPY-labeled CVN.

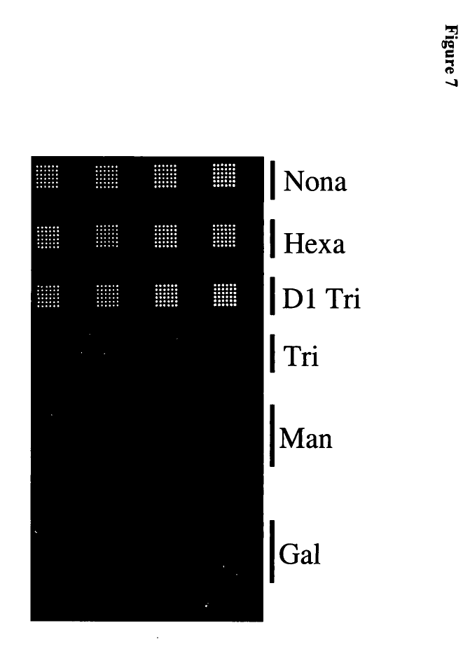

FIG. 8 depicts a high-mannose array printed with multiple concentrations of oligosaccharide subsequently incubated with BODIPY-labeled CVN.

FIG. 9 depicts a plot of intensity of CVN binding as a function of concentration of printed oligosaccharide.

FIG. 10 depicts glycoprotein immobilization on solid support.

FIG. 11 depicts a high-mannose array sequentially incubated with coumarin-labeled CVN followed by CY3-labeled 2G12.

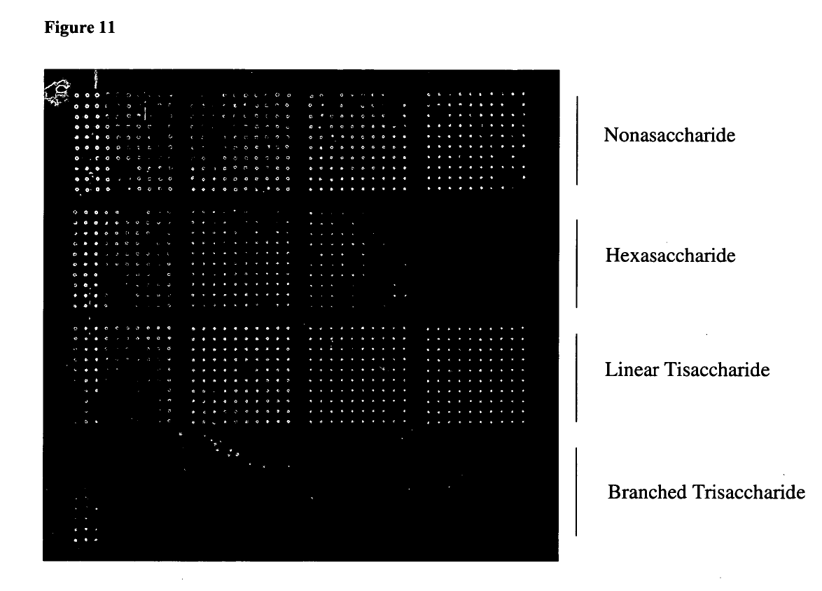

FIG. 12 depicts multiple experiments on a single chip utilizing Grace Bio-Labs silicone rubber gasket to divide chip into multiple incubation wells using high-mannose chip incubated with coumarin-labeled CVN and FITC-labeled ConA.

FIG. 13 depicts a hybrid array of high-mannose oligosaccharides and glycoproteins, screened against uncharacterized biotinylated extract using detection with FITC-labeled SAV.

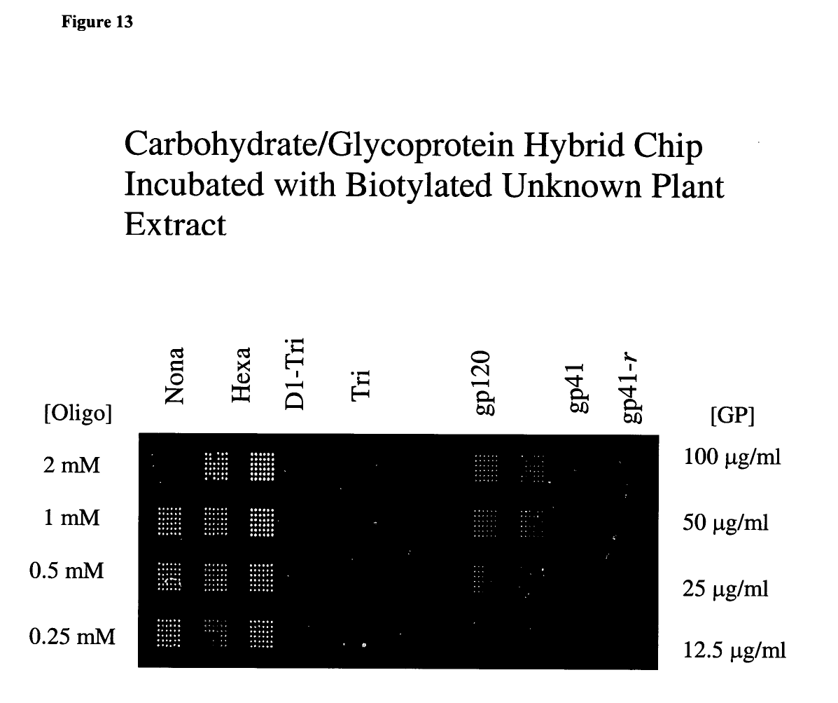

FIG. 14 depicts CVN and DC-SING binding profiles, screened against immobilized glycoproteins and neoglycocopeptides.

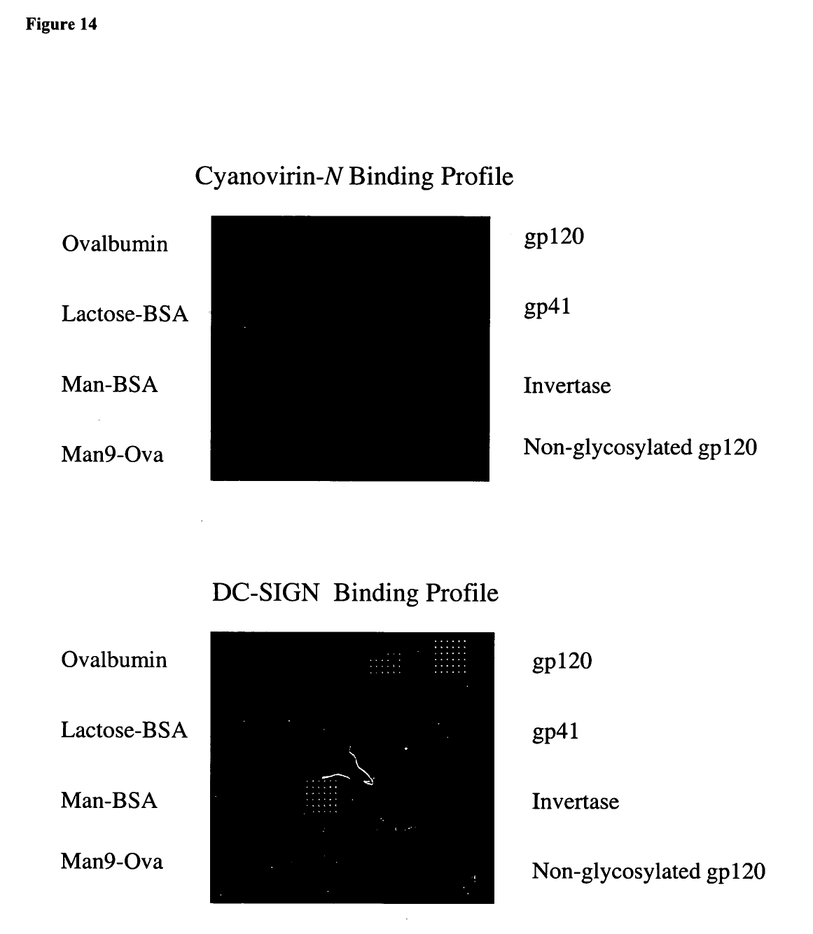

FIG. 15 depicts 2G12-N and CD4 binding profiles, screened against immobilized glycoproteins and neoglycocopeptides.

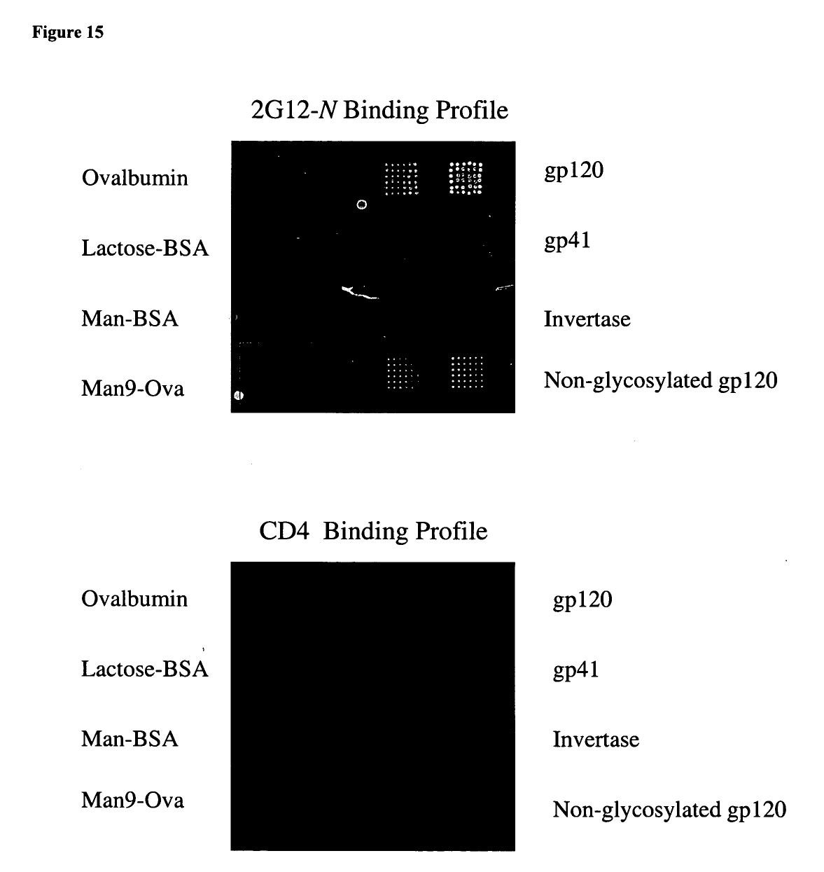

FIG. 16 depicts CD4/DC-SIGN sequential incubation binding profiles, screed against immobilized glycoproteins and neoglycocopeptides.

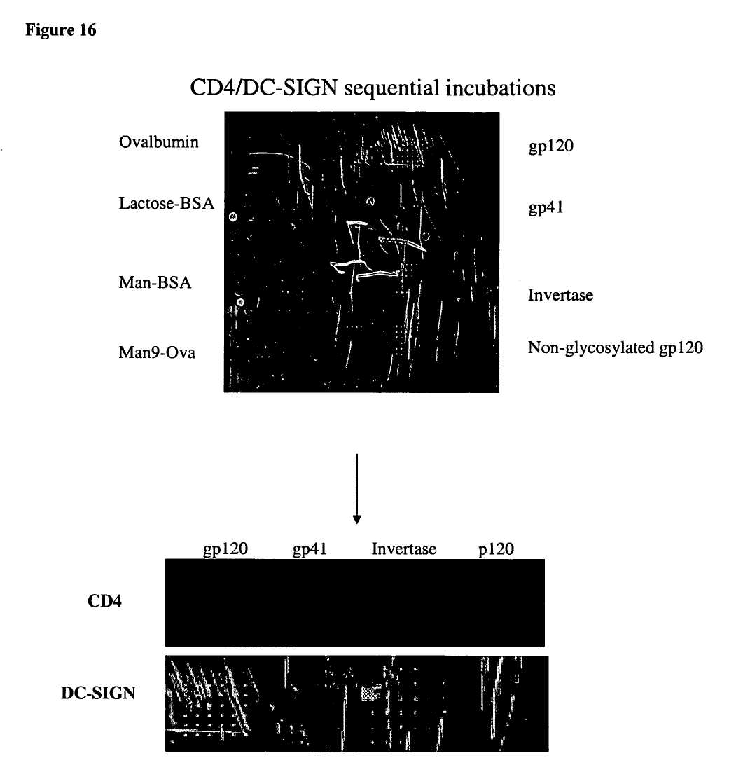

FIG. 17 depicts CD4/CVN sequential incubation binding profiles, screened against immobilized glycoproteins and neoglycocopeptides.

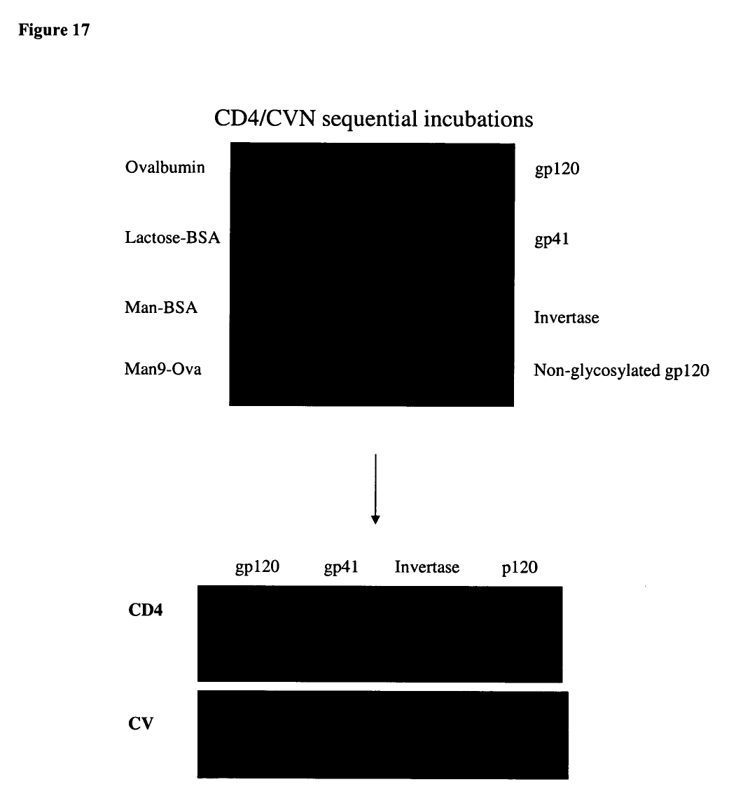

FIG. 18 depicts a plot of fluorescence intensity versus concentration of ConA for detection of mannose- and galactose-labeled beads.

FIG. 19 depicts a chart of fluorescence count for carbohydrate-labeled beads.

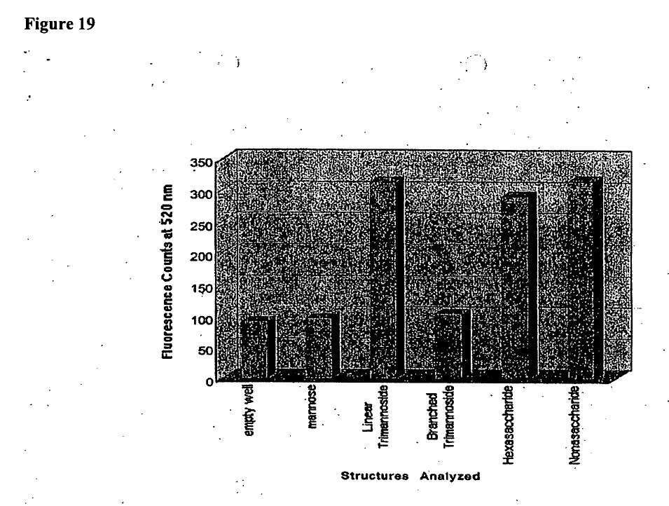

FIG. 20 depicts a carbohydrate-labeled bead experimental design.

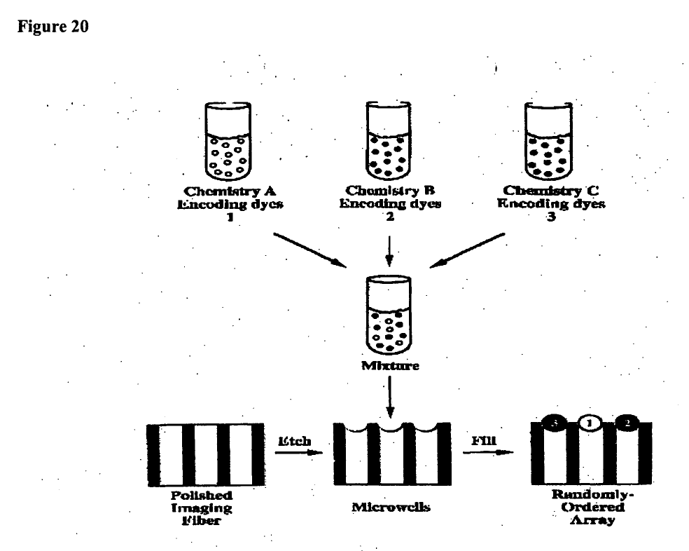

FIG. 21 depicts procedures to attach a reducing sugar to a triethyleneglycol linker.

FIG. 22 depicts procedures to attach a reducing sugar to a triethyleneglycol linker.

FIG. 23 depicts procedures to attach a reducing sugar to a triethyleneglycol linker.

### DETAILED DESCRIPTION OF THE INVENTION

The invention will now be described more fully with reference to the accompanying examples, in which certain preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Overview of a Preferred Embodiment

Assay miniaturization through the construction of high-density microarrays is particularly well suited to investigations in the field of glycomics. Unlike DNAs and proteins, which can be obtained in significant quantities through polymerase chain reaction amplification and cloning, respectively, no such ‘biological amplification’ strategy exists for the production of usable quantities of complex oligosaccharides. Consequently, investigators must rely upon arduous isolation techniques to derive oligosaccharides from natural sources or prepare these complex structures via chemical synthesis. Miniaturization of assay formats helps solve this fundamental problem by requiring only small quantities of material (pmol/array), enabling several experiments to be carried out on a single glass slide. This improved technology enables the efficient immobilization of oligosaccharides drawn from solution-phase synthesis, solid-phase synthesis—including automated solid-phase synthesis—and natural sources. Such carbohydrate arrays are useful tools in the identification of carbohydrate-protein interactions and help define the specific oligosaccharide structures involved in binding events. In addition, carbohydrate arrays are useful for rapid screening experiments to detect compounds that selectively inhibit protein-oligosaccharide interactions.

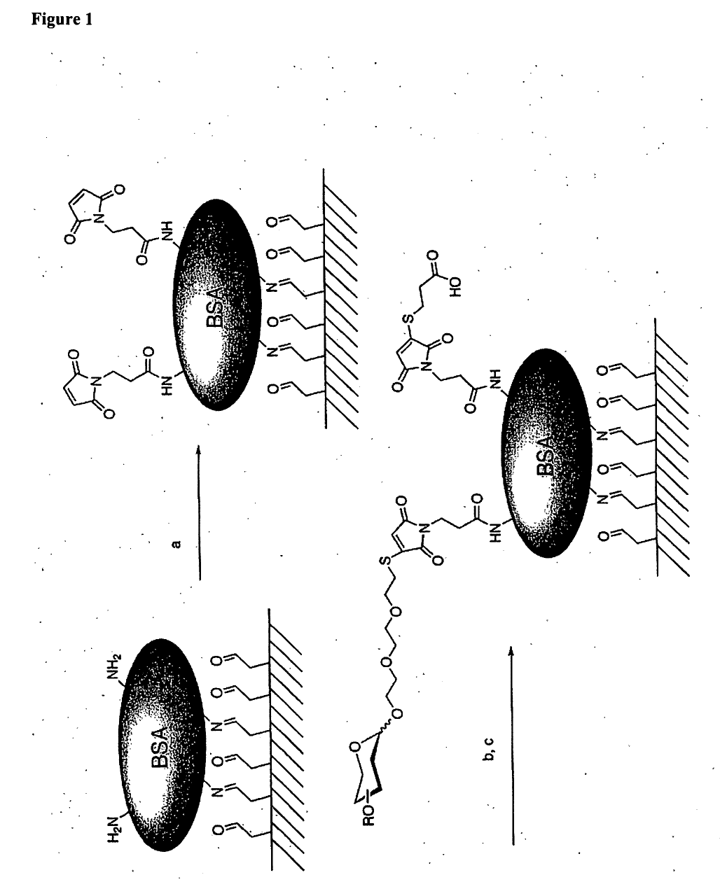

One aspect of the present invention relates to an array consisting of a plurality of spots, each comprising a carbohydrate molecule attached to a solid support. The carbohydrate molecule is a monosaccharide, oligosaccharide, polysaccharide, or glycoprotein. In certain preferred embodiments, the carbohydrate comprises mannose or galactose, or is glycoprotein gp120. The carbohydrate molecule may be attached to the solid support via a linker, e.g., bovine serum albumin (FIG. 1). The hydrophilic linker minimizes interactions between the array matrix and the solution phase proteins. In addition, the linker is compatible with a wide range of assay conditions, a limitation often encountered with noncovalent forms of attachment. In certain preferred embodiments, the carbohydrate molecule is attached to the linker by a sulfide bond. This procedure allows carbohydrate substrates drawn from solution-phase chemistry, solid phase chemistry, and natural sources to be readily incorporated into the present method. Furthermore, the carbohydrate may be bound to the solid support at varying concentration densities to permit determination of relative binding affinities. In other preferred embodiments, the array consists of spots that are about 120 μm in diameter and the distance between adjacent spots is about 300 μm. The high density of the arrays is advantageous because it requires only minute amount of carbohydrate substrate and is amenable to high-throughput technology.

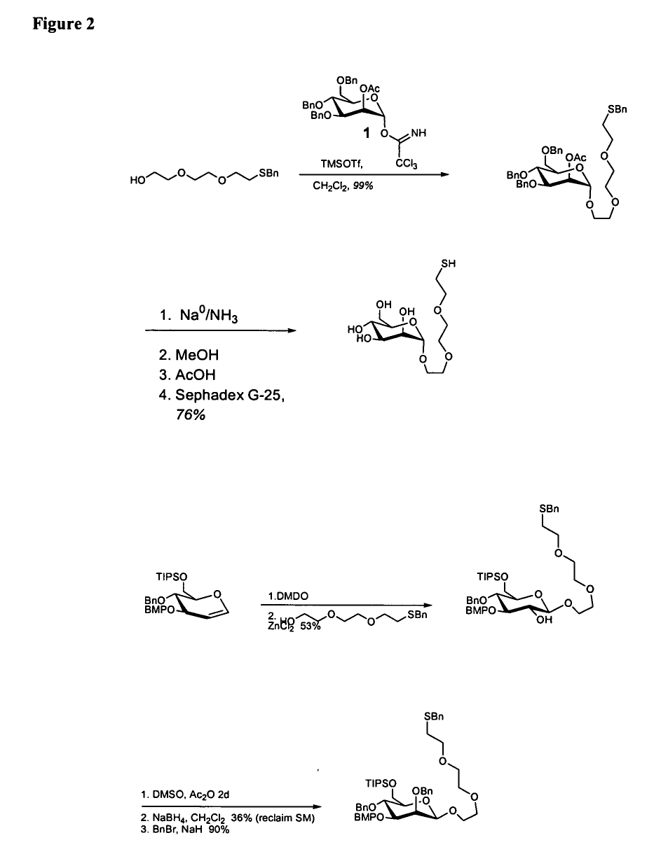

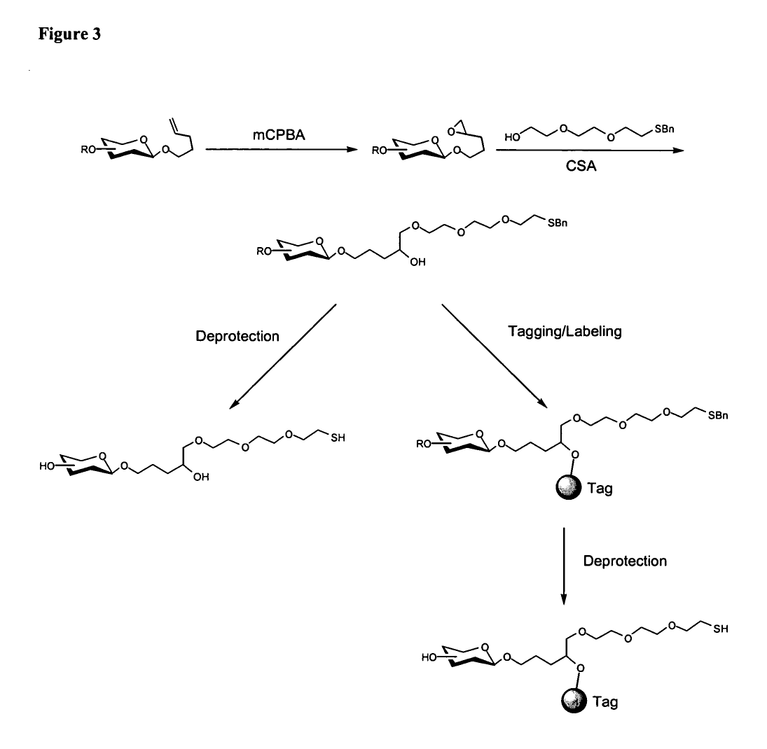

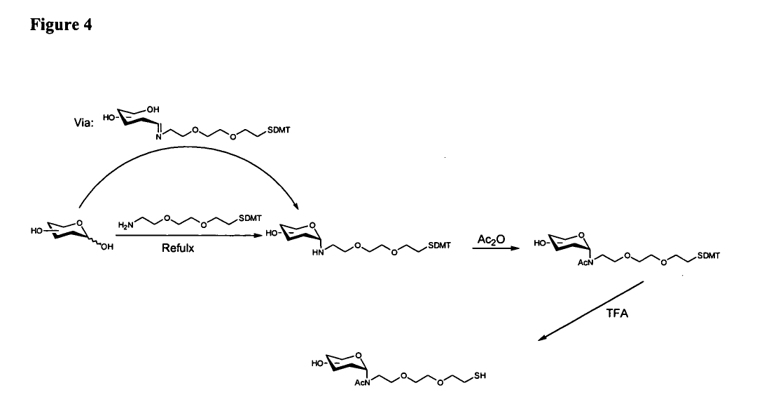

Another aspect of the present invention relates to a method of preparing an array of carbohydrate molecules, comprising the steps of applying a carbohydrate compound to a support to form a localized spot that is about 120 μm in diameter and a distance of about 300 μm from an adjacent spot. The carbohydrate molecule is a monosaccharide, oligosaccharide, polysaccharide, or glycoprotein. In certain preferred embodiments, the carbohydrate is mannose, galactose, oligomannose, or glycoprotein gp120. The carbohydrate molecule may be attached to the solid support via a linker, e.g. bovine serum albumin. In certain preferred embodiments, the carbohydrate molecule is attached to the linker by a sulfide bond. This mode of attachment is advantageous because it is amenable to preparation of carbohydrate microarrays wherein the carbohydrate is drawn from solution-phase chemistry (FIG. 2), solid-phase chemistry (FIG. 3), or natural sources (FIG. 4). In addition, the carbohydrate microarray may be prepared using precision printing robotics. The rapid construction of large arrays enables many carbohydrate binding experiments to be conducted efficiently and with little waste.

Another aspect of the present invention relates to a method to detect the interaction of a carbohydrate with a binding molecule, comprising the steps of contacting a binding molecule to a carbohydrate array and detecting the presence of a complex formed between the carbohydrate and binding molecule. The binding molecule is a protein, lipid, glycoprotein, DNA, RNA, or small organic molecule that contains a detectable functional group. In preferred embodiments, the binding molecule is detected by fluorescence spectroscopy, such as in BODIPY-labeled cyanoviron-N or FITC-labeled Concanavalin A. In other embodiments, multiple proteins are tagged with different fluorescent labels for use in binding competition experiments.

Another aspect of the present invention relates to a method to detect the interaction of a carbohydrate with a molecule of interest, comprising the steps of contacting a binding molecule to a carbohydrate bound to the surface of a microsphere and detecting the presence of a complex formed between the carbohydrate and binding molecule. The range of carbohydrate substrates and binding proteins discussed above for the carbohydrate microarray can be employed in the carbohydrate microsphere binding studies. In preferred embodiments, the binding event is detected by fluorescence spectroscopy. The microsphere may be composed of glass and optionally contain a signature dye to facilitate identification of the microsphere. In other preferred embodiments, the carbohydrate is bonded to the microsphere via a linker, such as bovine serum albumin wherein the carbohydrate molecule is bound to the linker by a sulfide bond.

The microarray of the present invention alleviates several of the problems associated with current microarray technology. First, the arrays of the present invention are printed at a high density, requiring little material for the manufacture of many hundreds of arrays (<pmol carbohydrate/array). This characteristic has obvious implications for reducing the costs of large-scale microarray production and also means that one can generate arrays with small amounts of precious structures (i.e., difficult to isolate or synthesize). These types of experiments would not be possible with non-miniaturized assay formats. Second, the arrays of the present invention are amenable to standard technologies used in high-throughput screening applications, such as high-density precision printing robotics and fluorescence scanning instrumentation. This amenability allows researchers who routinely perform DNA and protein array experiments to adopt carbohydrate array technology with minimal difficulty. In addition, immobilization chemistry of the present invention allows for structures of interest to be drawn from solution-phase synthesis, automated solid-phase synthesis, and natural sources (e.g., glycoproteins). This technology can also be applied to the preparation of ‘hybrid’ arrays consisting of both carbohydrate structures and glycoproteins immobilized on a single slide. Finally, immobilization chemistry of the present invention provides structures covalently immobilized on a hydrophilic non-fouling surface. Two major advantages of this technology are: a) the arrays are compatible with a wide range of assay conditions (e.g., wide pH range, detergent concentrations, ionic strength) whereas systems that make use of only hydrophobic or electrostatic interactions, are greatly limited in this regard; b) non-specific interactions between solution-phase proteins and the array matrix are greatly minimized, leading to high signal/noise ratios and little opportunity for ‘false positive’ results. A simple high throughput screening system will be of utmost importance to identify important carbohydrate-protein interactions and to find small molecules that block such interactions.

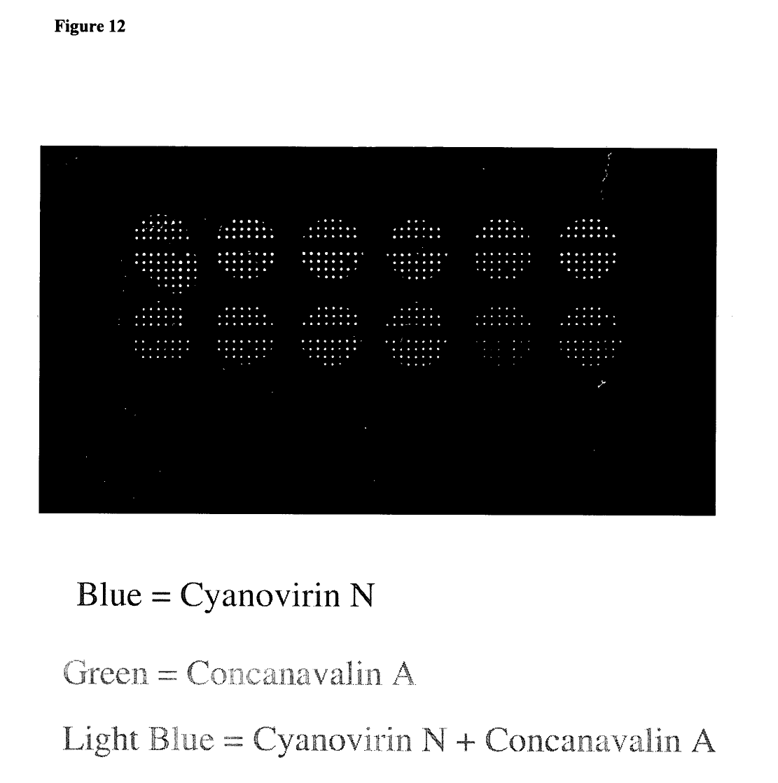

Above and beyond the features noted above, the carbohydrate array of the present invention has several additional technological advantages. First, multiple proteins can be tagged with different fluorescent labels to permit binding competition experiments. This capability has been demonstrated where a high-mannose array was sequentially incubated with coumarin-labeled CVN followed by CY3-labeled 2G12. Second, commercial silicone-rubber gaskets (Grace Bio-Labs) may be used to subdivide the field of a single array to perform multiple binding, competition or inhibition experiments simultaneously as illustrated in FIG. 12. In addition, multiple surface chemistries for the immobilization of both thiol-modified oligosaccharides and glycoproteins can be achieved on a single chip. Hybrid chips containing both carbohydrates and a binding protein can be prepared to investigate protein-carbohydrate binding. Such hybrid chips will be ideal tools to examine protein-carbohydrate and protein-glycoprotein interactions simultaneously. Finally, solid-support substrate may be modified to include a gold surface and remain amendable to high-density printing technology. This fact will enable array analysis by matrix-assisted laser desorption ionization mass spectrometry (MALDI) and surface plasmon resonance spectroscopy (SPR).

Microarrays Comprising Naturally Derived Reducing Sugars

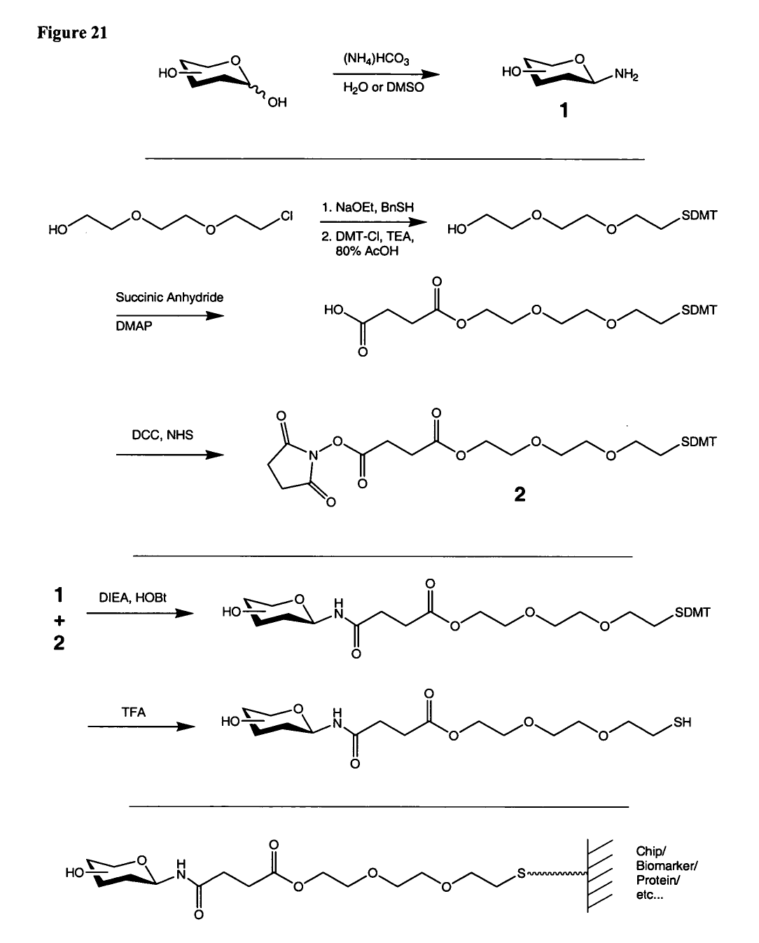

The arrays and methods described above can be adapted to comprise naturally derived reducing sugars. Based on the novel sulfhydryl containing ethylene glycol linker, we have developed methods to prepare microarrys of natural (non-synthetic) reducing sugars. The reducing sugar is converted to a glycosylamine and covalently bound to the triethyleneglycol linkers through an amide bond. The procedures for attaching the reducing sugar to the thiol-containing linker are displayed in FIGS. 21-23. It turns out that conversion of reducing sugars into their corresponding glycosylamines 1 is known (FIG. 1). See (a) Likhosherstov, L. M.; Novikova, 0. S.; Derevitskaja, V. A.; Kochetkov, N. K. Carb. Res. 1986, 146, C1; (b) Kallin, E.; Lonn, H.; Norberg, T. Elofsson, M. J. Carb. Chem. 1989, 8, 597; (c) Cohen-Anisfeld, S. T. and Lansbury, P. T. Jr. J. Am. Chem. Soc. 1993, 115, 10531; (d) Vetter, D. and Gallop, M. A. Bioconjugate Chem. 1995, 6, 316; and (e) Meinjohanns, E.; Meldal, M.; Paulsen, H.; Dwek, R. A.; Bock, Klaus. J. Chem. Soc., Perkin Trans. 1 1998, 3, 549. In addition, the reaction of glycosylamines with N-hydroxysuccinimide (NHS) activated esters has been used to covalently link a sulhydryl containing ethylene glycol linker to the glycosylamine of naturally procured carbohydrates through an amide bond. See Vetter, D.; Tate, E. M.; Gallop, M. A. Bioconjugate Chem. 1995, 6, 319.

The novel linker 2 incorporates the NHS-activated succinate of triethyleneglycol containing a masked terminal thiol moiety (See FIG. 21). Selected for its stability, and ease of deprotection, the dimethoxytrityl (DMT) protecting group serves to block the sulfhydryl of the linker. Upon incubation of the NHS-activated linker with glycosylamine in the presence of 1-hydroxybenzotriazole hydrate (HOBt) and diisopropylethylamine (DIEA), the sulfhydryl is deprotected by exposure to acid (TFA).

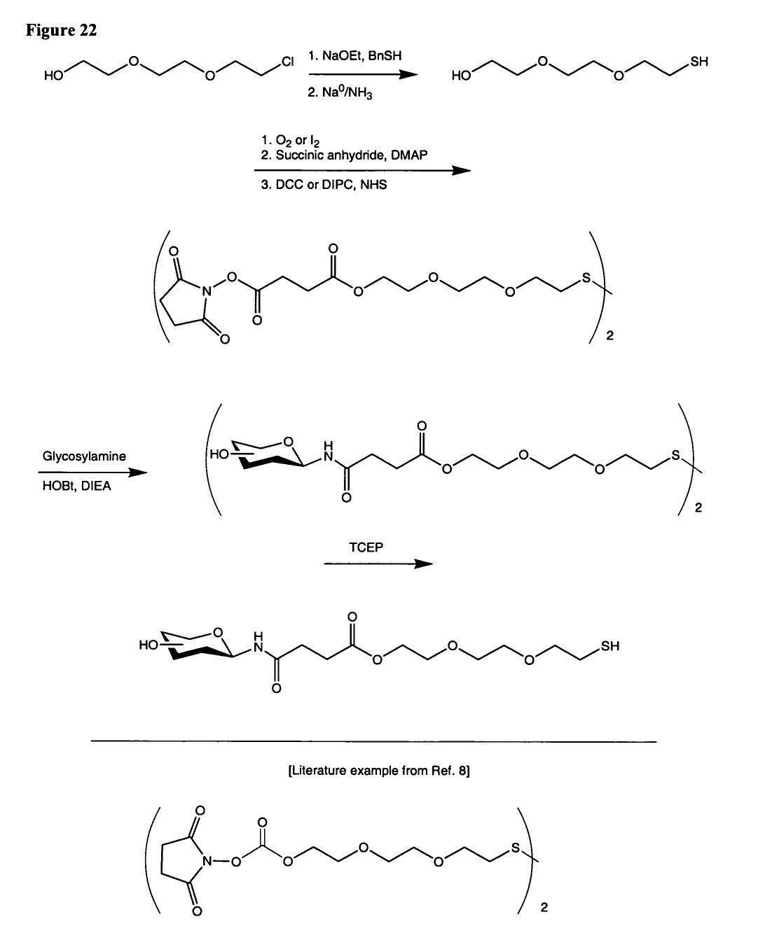

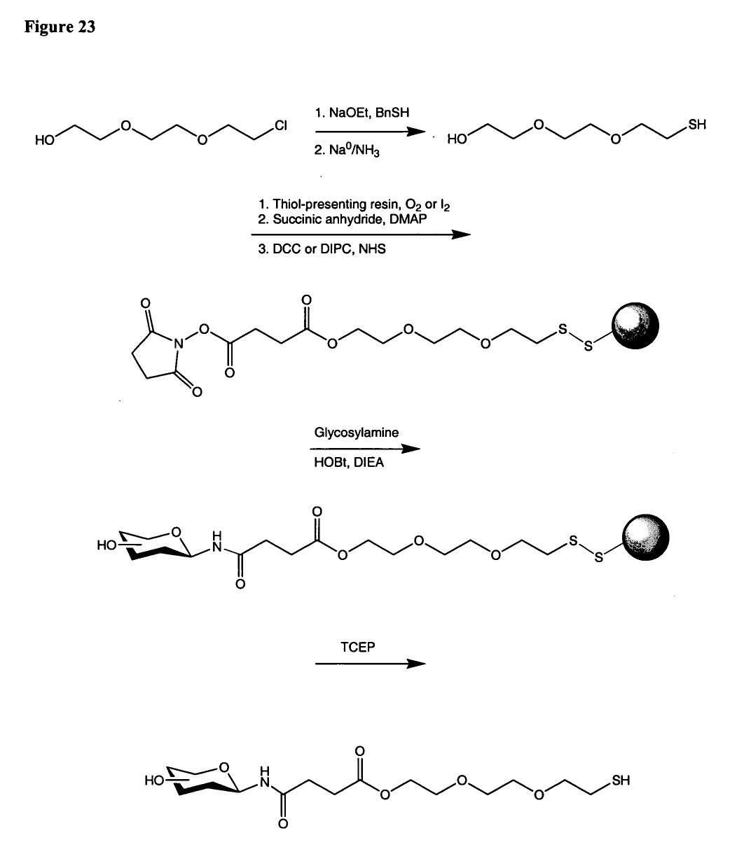

The disulfide that is formed upon the oxidation of the sulfhydryl containing triethyleneglycol in the presence of another thiol can be used to mask the sulhydryl moiety during intermediary reaction steps (FIG. 22). Using either a thiol-containing resin (See FIG. 23), or another molecule of sulfhydryl triethyleneglycol, the disulfide is readily formed upon oxidation using O₂, 12 or H₂O₂. See Lang, H.; Duschl, C.; Vogel H. Langmuir. 1994, 10, 197. Upon completion of all reactions, the sulfhydryl can be reconstituted by reduction with tris(2-carboxyethyl)-phosphine hydrochloride (TCEP) or appropriate reductants. A NHS activated ethylene glycol disulfide linker has been reported for coupling ethylene glycol linkers to amine containing biomolecules. See Boden, N.; Bushby, R. J.; Liu, Q.; Evans, S. D.; Jenkins, A. T. A.; Knowles, P. F.; Miles, R. E. Tetrahedron. 1998, 54, 11537.

Microarrays

A microarray may include any one-, two- or three-dimensional arrangement of addressable regions, or features, each bearing a particular chemical moiety or moieties, such as a carbohydrate, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present and do not carry probe molecules. Such interfeature areas are present where the arrays are formed by processes involving drop deposition of reagents, but may not be present when the photolithographic array fabrication processes are used.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m and a width of more than 4 mm and less than 1 m, although other shapes are possible as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50%, of the illuminating light incident on the front as may be measured across the entire integrated spectrum of such illuminating light.

Microarrays can be prepared that contain biopolymers, synthetic polymers, and other types of chemical entities. Biopolymers are typically found in biological systems and particularly include polysaccharides, peptides, and polynucleotides, as well as their analogs such as those compounds containing amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivatized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Microarrays that contain carbohydrate molecules have been reported. The carbohydrate can be bound to solid support by a covalent or noncovalent interaction. The mode of attachment is limited by the necessity that the bond formed between the solid support and the carbohydrate is both durable and does not interfer with testing assays, e.g. binding affinity or biological activity. See D. Schena et al. Science 1995, 270, 467 and S. L. Schreiber et al. Science 2000, 289, 176. In one example, nitrocellulose coated slides were employed for the noncovalent immobilization of microbial polysaccharides and neoglycolipid modified oligosaccharides S. Fukui et al. Nat. Biotechnol. 2002, 20, 1011. Wong and coworkers used hydrophobic interactions to anchor lipid-bearing carbohydrates onto polystyrene microtiter plates C. H. Wong al. J. Am. Chem. Soc. 2002, 124, 14397. This technology entailed forming triazole rings by 1,3-dipolar cycloaddition of alkynes and azides wherein the azido group was attached to the carbohydrate via an ethylene tether. The triazole ring functioned as a hydrophobic anchor to a solid support comprised of saturated hydrocarbon chains 13-15 carbons in length. However, noncovalent bonds are not as strong as a covalent bonds. In one approach to covalent attachment of a carbohydrate to a solid support, Diels-Alder-mediated covalent immobilization of cyclopentadiene-derivatized monosaccharides was achieved on a gold surface bearing benzoquinone groups B. Houseman and M. Mrksich Chem. Biol. 2002, 9, 443. Another covalent immobilization technology involved reacting maleimide functionalized mono- and di-saccharide glycosylamines with a thiol-derivatized glass slide, or alternatively, thiol-functionalized carbohydrates with a self-assembled monolayer presenting maleimide groups. See S. Park et al. Angew. Chem. Int. Ed. 2002, 41, 3180.

Method to Make Microarray

Microarrays of biological materials are comprised of a number of small discrete deposits of biological materials such as DNA, RNA, proteins, or carbohydrates, in predetermined patterns on a solid support. The support generally comprises glass, a polymer, or metal surface; however, glass supports can be coated with another material, e.g. gold. Gold covered surfaces would allow for direct analysis, by matrix-assisted laser desorption ionization mass spectrometry or surface plasmon resonance spectroscopy, of the material bound to the solid support. The biological material, e.g. protein or carbohydrate, may be bound to the solid support by through a covalent or non-covalent attachment. In some cases, a substrate can be bound to the solid support by a linker such as bovine serum albumin. In addition, automated technologies have been developed to simplify rapid assembly of microarrays.

Photolithography, mechanical microspotting, and ink jet technology have been used for the automated production of microarrays containing biomolecules. With photolithography, a glass wafer, modified with photolabile protecting groups is selectively activated by shining light through a photomask. This method has been used to prepared high-density oligonucleotide microarrays by repeated deprotection and coupling cycles. See U.S. Pat. No. 5,744,305. Microspotting encompasses deposition technologies that enable automated microarray production by printing small quantities of pre-made biochemical substances onto solid surfaces. Printing is accomplished by direct surface contact between the printing substrate and a delivery mechanism, such as a pin or a capillary. Robotic control systems and multiplexed printheads allow automated microarray fabrication. Ink jet technologies utilize piezoelectric and other forms of propulsion to transfer biochemical substances from miniature nozzles to solid surfaces. Using piezoelectricity, the sample is expelled by passing an electric current through a piezoelectric crystal which expands to expel the sample. Piezoelectric propulsion technologies include continuous and drop-on-demand devices. In addition to piezoelectric ink jets, heat may be used to form and propel drops of fluid using bubble-jet or thermal ink jet heads; however, such thermal ink jets are typically not suitable for the transfer of biological materials due to the heat can degrade biological samples. See U.S. Pat. No. 5,658,802.

Another method for making arrays of biological materials is called the “dot blot” approach. This method has been successfully employed for the production of DNA microarrays. In this method, a vacuum manifold transfers a plurality, e.g., 96, aqueous samples of DNA from 3 millimeter diameter wells to a porous membrane. The DNA is immobilized on the porous membrane by baking the membrane or exposing it to UV radiation. This is a manual procedure practical for making one array at a time and usually limited to 96 samples per array. “Dot-blot” procedures are therefore inadequate for applications in which many thousand samples must be determined. Another technique employed for making ordered arrays of genomic fragments uses an array of pins dipped into the wells, e.g., the 96 wells of a microtitre plate, for transferring an array of samples to a substrate, such as a porous membrane. One array includes pins that are designed to spot a membrane in a staggered fashion, for creating an array of 9216 spots in a 22×22 cm area. A limitation with this approach is that the volume of DNA spotted in each pixel of each array is highly variable. In addition, the number of arrays that can be made with each dipping is usually quite small.

A variety of chemically derivatized glass slides that can be printed on and imaged using commercially available arrayers and scanners may be used as a solid support for the microarrays. In certain embodiments, glass slides that have been treated with an aldehyde-containing silane reagent are used. In one embodiment of special interest, glass slides with aldehyde moieties attached are purchased from TeleChem International (Cupertino, Calif.) under the trade name “SuperAldehyde Substrates”. The aldehyde groups on the surface of these slides react readily with primary amines on the proteins to form a Schiffs base linkage. Since typical proteins display many lysine residues on their surface, as well as the generally more reactive α-amine at their N-terminus, they can attach to the slide in a variety of orientations, permitting different sides of the protein to interact with other proteins, small molecules, or small molecules in solution.

Binding Agents

In theory, the composition of the molecule that binds to the carbohydrate of interest need not be constrained to any one type, shape, or size of molecule. This comes as a result of the fact that intermolecular attraction can be caused by a variety of interactions including hydrogen bonding, ionic attraction, and hydrophobic affects. In fact, these types of molecular interactions between carbohydrates and other biomolecules are thought to play an important role in physiological processes. One such biomolecule is protein cyanovirin-N (CVN) B. O'Keefe, et al. Mol. Pharmacol. 2000, 58, 982. CVN was isolated from the blue-green algae Nostoc elliposporum and was found to bind the high-mannose oligosaccharides of gp120, thereby inhibiting HIV's ability to infect target cells. A. Bolmstedt et al. Mol. Pharmacol. 2001, 59, 949. Natural and recombinant forms of CVN have been shown to irreversibly inactivate a wide variety of HIV strains while exhibiting minimal toxicity to host cells. Boyd, K et al. Antimicrob. Agents Chemother. 1997, 41, 1521. The ability of CVN to bind high-mannose oligosaccharides make it an ideal test case for a carbohydrate array containing synthetic oligosaccharides of different lengths and complexity. Furthermore, the CVN can be modified to contain a fluorescent tag, e.g. BODIPY, to facilitate detection of the carbohydrate bound CVN. Other suitable substrates for carbohydrate recognition studies include FITC-labeled Concanavalin A, Texas Red-labeled Erythrina cristagalli (ECA), DC-SIGN, 2G12-N, CD4, FITC-SAV, or CY3-labeled 2G12.

Microarray Analysis Assay

The develop of microarrays comprising a large number of experiments necessitates a detection method that is sensitive, selective, and rapid. One approach to monitoring microarray experiments employs radiometric or optical analysis. Radiometric or optical analysis produces a scanned image consisting of a two-dimensional matrix of pixels, each pixel having one or more intensity values corresponding to one or more signals. Scanned images are commonly produced electronically by optical or radiometric scanners and the resulting two-dimensional matrix of pixels is stored in computer memory or on a non-volatile storage device. Alternatively, analog methods of analysis, such as photography, can be used to produce continuous images of a microarray that can be then digitized by a scanning device and stored in computer memory or in a computer storage device.

The results of microarray experiments can be detected by fluorescence spectroscopy. Fluorescence is a physical phenomenon based upon the ability of some molecules to absorb light (photons) at specified wavelengths and then emit light of a longer wavelength and at a lower energy. Substances able to fluoresce share a number of common characteristics: the ability to absorb light energy at one wavelength; reach an excited energy state; and subsequently emit light at another light wavelength. The absorption and fluorescence emission spectra are individual for each fluorophore and are often graphically represented as two separate curves that are overlapping. The same fluorescence emission spectrum is generally observed irrespective of the wavelength of the exciting light and, accordingly, the wavelength and energy of the exciting light may be varied within limits; but the light emitted by the fluorophore will always provide the same emission spectrum. Finally, the strength of the fluorescence signal may be measured as the quantum yield of light emitted. The fluorescence quantum yield is the ratio of the number of photons emitted in comparison to the number of photons initially absorbed by the fluorophore. For more detailed information regarding each of these characteristics, the following references are recommended: Lakowicz, J. R., Principles of Fluorescence Spectroscopy, Plenum Press, New York, 1983; Freifelder, D., Physical Biochemistry, second edition, W. H. Freeman and Company, New York, 1982; “Molecular Luminescence Spectroscopy Methods and Applications: Part I” (S. G. Schulnan, editor) in Chemical Analysis, vol. 77, Wiley & Sons, Inc., 1985; The Theory of Luminescence, Stepanov and Gribkovskii, Iliffe Books, Ltd., London, 1968.

Fiber Optic Microsphere Arrays

Microspheres are spherically shaped objects that generally have a diameter less than 1 millimeter. Microspheres are commonly prepared from glass, polymers, or resins. However, microspheres can be made from a vast range of materials such as methylstyrene, polystyrene, acrylic polymer, latex, paramagnetic, thoria sol, carbon graphite, and titanium dioxide. See “Microsphere Detection Guide” from Bangs Laboratories, Fishers Ind. for a more complete listing. In addition, microspheres have been prepared that contain an internal fluorophore to enable detection of the microsphere. The surface of the microsphere can also be modified to contain a desired chemical moiety such as a DNA fragment, protein, or carbohydrate. This modification enables one to conduct binding experiments between the material bonded to the microsphere and a given analyte.

Steemers et al. describe an experiment wherein random fiber optic microsphere arrays were used for DNA hybridization detection. See F. J. Steemers et al. Nature Biotechnology 2000, 18, 91. The arrays comprised different populations of 4.5 μm microspheres that each contained their own internally encoded spectral signature (an entrapped fluorescent dye) and a unique carbohydrate structure covalently attached to its surface. The internal dye served two purposes in this experiment: it identified the carbohydrate present on the bead surface and aided determination of the position of each type of microsphere in the array.

U.S. Pat. No. 6,023,540 describes a microsphere-based analytical chemistry system in which microspheres carrying different chemical functionalities may be mixed together while the ability to identify the functionality on each bead is retained. This process entailed using an optically interrogatable encoding scheme comprised of incorporating dyes into the microsphere core. One aspect of the patent concerns a population of beads wherein the population contains separate subpopulations, each of which carries chemical functionality which changes the optical signature of the beads in a presence of targeted analytes. This signature change can occur via many different mechanisms. A few examples include the binding of a dye-tagged analyte to the bead, the production of a dye species on or near the beads, the destruction of an existing dye species, a change in optical signal upon analyte interaction with dye on bead, or any other optical interrogatable event. Although the subpopulations may be randomly mixed together, the chemical functionality on each bead is determined via an optical signature which is encoded with a description of the chemical functionality. As a result, by observing whether the optical signature of a particular bead is exhibiting a change, or not, and then decoding the signature for the functionality of the bead, the presence of the analyte targeted by the functionality may be determined. In certain examples, the beads are encoded using dyes that are preferably entrapped within the beads, the chemical functionality being added on surfaces. The dyes may be chromophores or phosphors but are preferably fluorescent dyes, which due to their strong signals provide a good signal-to-noise ratio for decoding. The encoding can be accomplished in a ratio of at least two dyes, although more encoding dimensions may be added in the size of the beads, for example.

It is noteworthy that microspheres may be purchased with a variety chemical functionalities already present. A large selection of such pre-prepared microspheres are currently available from a number of commercial vendors. Alternatively, “blank” microspheres may be used that have surface chemistries that facilitate the attachment of the desired functionality by the user. Some examples of these surface chemistries for blank microspheres are listed in Table I. Materials can also be bound to the surface of a microsphere through a linker such as bovine serum albumin.

Techniques for immobilizing enzymes on microspheres have been reported. In one case, NH₂ surface chemistry microspheres are used. Surface activation is achieved with a 2.5% glutaraldehyde in phosphate buffered saline (10 mM) providing a pH of 6.9. (138 mM NaCl, 2.7 mM, KCl). This is stirred on a stir bed for approximately 2 hours at room temperature. The microspheres are then rinsed with ultrapure water plus 0.01% between 20 (surfactant)-0.02%, and rinsed again with a pH 7.7 PBS plus 0.01% between 20. Finally, the enzyme is added to the solution, preferably after being prefiltered using a 0.45 μm amicon micropure filter.

TABLE I

| Surface chemistry | Name: |
|---|---|
| NH₂ | Amine |
| COOH | Carboxylic Acid |
| CHO | Aldehyde |
| CH₂—NH₂ | aliphatic Amine |
| CO NH₂ | Amide |
| CH₂—Cl | Chloromethyl |
| CONH—NH₂ | Hydrazide |
| OH | Hydroxyl |
| SO₄ | Sulfate |
| SO₃ | Sulfonate |
| Ar NH₂ | Aromatic Amine |

Microspheres can be labeled with chemical dyes to enable detection. Dyes may be covalently bonded to the microspheres' surface, but this consumes surface binding sites desirably reserved for the chemical functionalities. Preferably, the microspheres are placed in a dye solution comprising a ratio of two or more fluorescent reporter dyes dissolved in an organic solvent that will swell the microspheres, e.g., dimethylformamide (DMF). The length of time the microspheres are soaked in the dye solution will determine their intensity and the broadness of the ratio range. Longer times yield higher intensities, but broader ratio ranges.

In an example reported in U.S. Pat. No. 6,023,540, the dye Texas Red Cadaverine (TRC) was used, which is excited at λ_{ab}=580 mm and emits at λₑₘ=630 mm, in combination with indodicarbocyanine (DiIC): 610/670 (λ_{ab}/λₑₘ). Generally, dyes are selected to be compatible with the chemistries involved in the analysis and to be spectrally compatible. The emission wavelengths of the dyes should not overlap the regions of the optical spectrum in which the chemical functionalities induce changes in the microsphere signatures. This avoids deconvolution problems associated with determining signal contributions based on the presence of both the analyte and the encoding dye ratios contributing to an overlapping emission spectral region. Examples of other dyes that can be used are Oxazin (662/705), IR-144 (745/825), IR-140 (776/882), IR-125 (786/800) from Exiton, and Bodipy 665/676 from Molecular Probes, and Naphthofluorescein (605/675) also from Molecular Probes. Lanthide may also be used. Fluorescent dyes emitting in other than the near infrared may also be used. Chromophore dyes are still another alternative that produce an optically interrogatable signature, as are more exotic formulations using Raman scattering-based dyes or polarizing dyes, for example.

The ability of a particular dye pair to encode for different chemical functionalities depends on the resolution of the ratiometric measurement. Conservatively, any dye pair should provide the ability to discriminate at least twenty different ratios.

After the microsphere has been exposed to the dye the microspheres are vacuum filtered to remove excess dye. The microspheres are then washed in water or other liquid that does not swell the microspheres, but in which the dyes are still soluble. This allows the residual dye to be rinsed off without rinsing the dye out of the microspheres. Then, the chemical functionality is attached to the microsphere surface chemistries if not already present.

Techniques for immobilizing enzymes on microspheres are known. In one case, NH₂ surface chemistry microspheres are used. Surface activation is achieved with a 2.5% glutaraldehyde in phosphate buffered saline (10 mM) providing a pH of 6.9. (138 mM NaCl, 2.7 mM, KCl). This is stirred on a stir bed for approximately 2 hours at room temperature. The microspheres are then rinsed with ultrapure water plus 0.01% and with a pH 7.7 PBS solution. Finally, the enzyme is added to the solution, preferably after being prefiltered using a 0.45 μm amicon micropure filter.

The microspheres exhibiting activity or changes in their optical signature may be identified by utilizing a somewhat “manual” approach of observing the individual microspheres through a microscope. Decoding can also be performed manually, depending on the particular reporter dyes used. It may be helpful to use optical aids such as light filters to observe the light from the microspheres at emission wavelengths of the reporter dyes. While this approach is possible, in the preferred embodiment, the analytic chemistry microsphere system is used with the inventive optical fiber sensor.

Glycoproteins

Glycoproteins are those amino acid sequences that have a carbohydrate molecule covalently bound to an amino acid of the amino acid sequence. One example is gp120, an adhesion protein located on the envelope (surface membrane) of HIV (i.e., AIDS-causing) viruses that directly interacts with the CD4 protein on helper T cells; enabling the HIV viruses to bind to and infect helper T cells. A related protein is gp41. Glycoprotein gp41 is transmembrane protein associated with HIV. A more thorough account can be found in Clinical Virology 2^{nd} Ed., Ed. Richman, D. D.; Whitley, R. J.; Hayden, F. G. ASM Press: Washington, D.C., 2002.

Enzymatic Modification of Carbohydates

Many classes of enzymes are commercially available (e.g., Calbiochem) that can react with carbohydrate molecules. These enzymes often have different substrate specificity or carry out a different chemical reaction. One class of enzymes suitable for carbohydrate modification is glycosyltransferases. Glycosyltransferases can be subdivided into several smaller groups including fucosyltransferases, galactosyltransferases, mannosyltransferases, and sialyltransferases. Representative examples of fucosyltransferases include N-Acetyllactosamine-α1,3-L-fucosyltransferase V, Fuc-TV, Fuc-TVI, α1,3-Fucosyltransferase V, α1,3-Fucosyltransferase VI, and β-D-Galactosyl-β1,4-N-acetylglucosamine-α1,3-L-fucosyltransferase VI. Representative examples of galactosyltransferases include α1,3-Galactosyltransferase, N-Acetyl-β-D-glucosamine-β 1,4-D-galactosyltransferase, β-D-Galactosyl-N-acetylglucosamine-α1,3-D-galactosyltransferase, P 1,4-Galactosyltransferase, α1,3-Galactosyltransferase, α1,3-GalT, and β1,4-GalT. Representative examples of mannosyltransferases include α1,2-Mannosyltransferase, ManT, and Mnt1p. Representative examples of sialyltransferases include β-D-Galactosyl-β1,3/4-N-acetyl-β-D-glucosamine-α2,3-sialyltransferase, β-D-Galactosyl-β1,3-N-acetyl-β-D-galactosamine-α2,3-sialyltransferase, β-D-Galactosyl-β1,4-N-acetyl-β-D-glucosamine-α2,6-sialyltransferase, α2,3-NST, α2,6-NST, α2,3-OST, α2,6-(N)-Sialyltransferase, α2,3-(N)-Sialyltransferase, α2,3-(O)-Sialyltransferase, and Anti-ST3Gal III.

A second class of enzymes suitable for carbohydrate modification is exoglycosidases. Exoglycosidases can be subdivided into several smaller groups including fucosidases, galactosidases, hexosaminidases, hexosidases, mannosidases, neuraminidases, and xylosidases. Representative examples of fucosidases include α1,2-Fucosidase, α1-3,4-Fucosidase, α1,6-Fucosidase, α-L-Fucoside Fucohydrolase, α-L-Fucoside Fucohydrolase, and α-L-Fucoside Fucohydrolase. Representative examples of galactosidases include β-D-Galactoside galactohydrolase, β1,4-D-Galactoside Galactohydrolase, β1,6-D-Galactoside Galactohydrolase, β1-3,6-D-Galactoside Galactohydrolase, β-Galactosidase, α1-3,6-Galactosidase, β1,3-Galactosidase, β1-3,6-Galactosidase, β1,4-Galactosidase, β1,6-Galactosidase, and α1-3,6-D-Galactoside Galactohydrolase. Representative examples of hexosaminidases include N-Acetyl-β-D-glycosaminide-N-acetylglucosaminohydrolase, α-D-N-Acetylgalactosaminidase, α-N-Acetylgalactosaminidase, β1-2,3,4,6-N-Acetylglucosaminidase, β-N-Acetylhexosaminidase, and Hexosaminidase. Representative examples of hexosidases include β-Glycosidase I and β-Glycosidase II. Representative examples of mannosidases include α1-2,3-Mannosidase, α1-2,3,6-Mannosidase, α1,6-Mannosidase, β1,4-Mannosidase, and α-D-Mannoside Mannohydrolase. Representative examples of neuraminidases include Acetylneuraminyl Hydrolase, N-Acetylneuraminyl Hydrolase, Acetylneuraminyl Hydrolase Agarose, α2-3,6,8,9-Neuraminidase, α2,3-Neuraminidase, α2-3,6-Neuraminidase, α2-3,6,8-Neuraminidase, Sialidase, and Sialidase L. Representative examples of xylosidases include β-D-Xylanxylohydrolase, Exo-1,4-β-D-Xylodase, 1,4-β-D-Xylanxylohydrolase, βXYLase, β1,2-Xylosidase, and β1,4-Xylosidase.

A third class of enzymes suitable for carbohydrate modification is endoglycosidases. Representative examples of endoglycosidases include β-Endo-chitinase, Ceramide Glycanase, rEGCase II, EGCase II ACT, Endo F1, Endo F2, Endo F3, Endo H, Endo-β-galactosidase, Endo-α-N-acetylgalactosaminidase, Endo-β-N-acetylglucosaminidase F 1, Endo-β-N-acetylglucosaminidase F2, Endo-β-N-acetylglucosaminidase F3, Endo-β-N-acetylglucosaminidase H, Endoglycoceramidase II ACT, Endoglycoceramidase II, Endoglycosidase F1, Endoglycosidase F2, Endoglycosidase F3, Endoglycosidase H, Glycopeptidase A, Glycopeptidase F, Glycopeptidase F, O-Glycopeptide endo-D-galactosyl-N-acetyl-α-galactosaminohydrolase, O-Glycosidase, N-Glycosidase A, N-Glycosidase F, N-Glycosidase F, Oligoglycosylglucosylceramideglycohydrolase, Oligoglycosylglucosylceramide-glycohydrolase activator II, Peptide-N-⁴-(acetyl-β-glucosaminyl)-asparagine Amidase, Peptide-N-glycosidase F, PNGase A, PNGase F, and PNGase F.

Definitions

For convenience, certain terms employed in the specification, examples, and appended claims are collected here.

The articles “a” and “an” are used herein to refer to one or to more than one (i.e. to at least one) of the grammatical object of the article. By way of example, “an element” means one element or more than one element.

The term “microsphere” refers to an object that is substantially spherical in shape and has a diameter less than 1 millimeter.

The term “glass” refers to a hard, brittle, non-crystalline, inorganic substance, which is usually transparent; glasses are often made by fusing silicates with soda, as described by Webster's New World Dictionary. Ed. Guralnik, DB 1984.

The term “oligosaccharide” refers to three to ten sugar molecules linked by glycosidic bonds as described in Organic Chemistry 2^{nd} Ed. Ed. Bruice, P. Y. New Jersey: Prentice Hall, 1998.

The term “polysaccharide” refers to compound containing ten or more sugar molecules linked together as described in Organic Chemistry 2^{nd} Ed. Ed. Bruice, P. Y. New Jersey: Prentice Hall, 1998.

For purposes of this invention, the chemical elements are identified in accordance with the Periodic Table of the Elements, CAS version, Handbook of Chemistry and Physics, 67th Ed., 1986-87, inside cover.

Arrays of the Invention

One aspect of the present invention relates to an array, comprising a plurality of spots on a solid support, wherein each spot independently comprises a substrate attached to said solid support, wherein each substrate attached to said solid support is independently a carbohydrate-containing molecule.

In certain embodiments, the present invention relates to the aforementioned array, wherein said solid support is glass, gold-coated glass, polymer, or metal surface.

In certain embodiments, the present invention relates to the aforementioned array, wherein said solid support is glass or gold-coated glass.

In certain embodiments, the present invention relates to the aforementioned array, wherein said solid support is glass.

In certain embodiments, the present invention relates to the aforementioned array, wherein said carbohydrate-containing molecule is a monosaccharide, disaccharide, oligosaccharide, polysaccharide, glycoprotein, neoglycopeptide, or glycolipid.

In certain embodiments, the present invention relates to the aforementioned array, wherein said carbohydrate-containing molecule is a monosaccharide, disaccharide, oligosaccharide, or polysaccharide.

In certain embodiments, the present invention relates to the aforementioned array, wherein said carbohydrate-containing molecule is a monosaccharide or oligosaccharide.

In certain embodiments, the present invention relates to the aforementioned array, wherein said carbohydrate-containing molecule is a monasaccharide, trisaccharide, or hexasaccharide.

In certain embodiments, the present invention relates to the aforementioned array, wherein said carbohydrate-containing molecule is a monosaccharide.

In certain embodiments, the present invention relates to the aforementioned array, wherein said carbohydrate-containing molecule is mannose, galactose, lactose, or Man9.

In certain embodiments, the present invention relates to the aforementioned array, wherein said carbohydrate-containing molecule is mannose or galactose.

In certain embodiments, the present invention relates to the aforementioned array, wherein said carbohydrate-containing molecule is mannose.

In certain embodiments, the present invention relates to the aforementioned array, wherein said carbohydrate-containing molecule is a glycoprotein or neoglycopeptide.

In certain embodiments, the present invention relates to the aforementioned array, wherein said carbohydrate-containing molecule is gp120, gp41, gp41-r, invertase, or non-glycosylated g120.

In certain embodiments, the present invention relates to the aforementioned array, wherein said carbohydrate-containing molecule is gp120 or gp41.

In certain embodiments, the present invention relates to the aforementioned array, wherein said carbohydrate-containing molecule is attached to said solid support by a non-covalent interaction.

In certain embodiments, the present invention relates to the aforementioned array, wherein said carbohydrate-containing molecule is attached to said solid support by a covalent bond.

In certain embodiments, the present invention relates to the aforementioned array, wherein said carbohydrate-containing molecule is attached to said solid support by a linker.

In certain embodiments, the present invention relates to the aforementioned array, wherein said carbohydrate-containing molecule is attached to said linker by a sulfide bond.

In certain embodiments, the present invention relates to the aforementioned array, wherein said carbohydrate-containing molecule is attached to said linker though a glycosidic linkage.

In certain embodiments, the present invention relates to the aforementioned array, wherein said linker is bovine serum albumin.

In certain embodiments, the present invention relates to the aforementioned array, wherein the diameter of said spots is less than about 300 μm.

In certain embodiments, the present invention relates to the aforementioned array, wherein the diameter of said spots is less than about 200 μm.

In certain embodiments, the present invention relates to the aforementioned array, wherein the diameter of said spots is less than about 120 μm.

In certain embodiments, the present invention relates to the aforementioned array, wherein the diameter of said spots is less than about 80 μm.

In certain embodiments, the present invention relates to the aforementioned array, wherein the distance between adjacent spots is less than about 900 μm.

In certain embodiments, the present invention relates to the aforementioned array, wherein the distance between adjacent spots is less than about 500 μm.

In certain embodiments, the present invention relates to the aforementioned array, wherein the distance between adjacent spots is less than about 300 μm.

In certain embodiments, the present invention relates to the aforementioned array, wherein the distance between adjacent spots is less than about 150 μm.

In certain embodiments, the present invention relates to the aforementioned array, wherein the diameter of said spots is less than about 120 μm and the distance between adjacent spots is less than about 300 μm.

In certain embodiments, the present invention relates to the aforementioned array, wherein said spots comprise said carbohydrate-containing molecule and at least one protein.

In certain embodiments, the present invention relates to the aforementioned array, wherein said array is subdivided into sections using a silicone-rubber gasket.

In certain embodiments, the present invention relates to the aforementioned array, wherein said array comprises a first collection of spots having a first concentration of said carbohydrate-containing molecule, and a second collection of spots having a second concentration of said carbohydrate-containing molecule.

Methods for Fabrication of Array

Another aspect of the present invention relates to a method of preparing an array of carbohydrate-containing molecules, comprising the step of:
applying a carbohydrate-containing molecule to a solid support to form a spot that has a diameter less than about 500 μm.

In certain embodiments, the present invention relates to the aforementioned method, wherein said solid support is glass, gold-coated glass, polymer, or metal surface.

In certain embodiments, the present invention relates to the aforementioned method, wherein said solid support is glass or gold-coated glass.

In certain embodiments, the present invention relates to the aforementioned method, wherein said solid support is glass.

In certain embodiments, the present invention relates to the aforementioned method, wherein said carbohydrate-containing molecule is a monosaccharide, disaccharide, oligosaccharide, polysaccharide, glycoprotein, neoglycopeptide, or glycolipid.

In certain embodiments, the present invention relates to the aforementioned method, wherein said carbohydrate-containing molecule is a monosaccharide, disaccharide, oligosaccharide, or polysaccharide.

In certain embodiments, the present invention relates to the aforementioned method, wherein said carbohydrate-containing molecule is a monosaccharide or oligosaccharide.

In certain embodiments, the present invention relates to the aforementioned method, wherein said carbohydrate-containing molecule is a monasaccharide, trisaccharide, or hexasaccharide.

In certain embodiments, the present invention relates to the aforementioned method, wherein said carbohydrate-containing molecule is a monosaccharide.

In certain embodiments, the present invention relates to the aforementioned method, wherein said carbohydrate-containing molecule is mannose, galactose, lactose, or Man9.

In certain embodiments, the present invention relates to the aforementioned method, wherein said carbohydrate-containing molecule is mannose or galactose.

In certain embodiments, the present invention relates to the aforementioned method, wherein said carbohydrate-containing molecule is mannose.

In certain embodiments, the present invention relates to the aforementioned method, wherein said carbohydrate-containing molecule is a glycoprotein or neoglycopeptide.

In certain embodiments, the present invention relates to the aforementioned method, wherein said carbohydrate-containing molecule is gp120, gp41, gp41-r, invertase, or non-glycosylated g120.

In certain embodiments, the present invention relates to the aforementioned method, wherein said carbohydrate-containing molecule is gp120 or gp41.

In certain embodiments, the present invention relates to the aforementioned method, wherein said carbohydrate-containing molecule is attached to said solid support by a noncovalent interaction.

In certain embodiments, the present invention relates to the aforementioned method, wherein said carbohydrate-containing molecule is attached to said solid support by a covalent bond.

In certain embodiments, the present invention relates to the aforementioned method, wherein said carbohydrate-containing molecule is attached to said solid support by a linker.

In certain embodiments, the present invention relates to the aforementioned method, wherein said carbohydrate-containing molecule is attached to said linker by a sulfide bond.

In certain embodiments, the present invention relates to the aforementioned method, wherein said carbohydrate-containing molecule is attached to said linker though a glycosidic linkage.

In certain embodiments, the present invention relates to the aforementioned method, wherein said linker is bovine serum albumin.

In certain embodiments, the present invention relates to the aforementioned method, wherein the diameter of said spots is less than about 300 μm.

In certain embodiments, the present invention relates to the aforementioned method, wherein the diameter of said spots is less than about 200 μm.

In certain embodiments, the present invention relates to the aforementioned method, wherein the diameter of said spots is less than about 120 μm.

In certain embodiments, the present invention relates to the aforementioned method, wherein the diameter of said spots is less than about 80 μm.

In certain embodiments, the present invention relates to the aforementioned method, wherein the distance between adjacent said spots is less than about 300 μm.

In certain embodiments, the present invention relates to the aforementioned method, wherein the distance between adjacent said spots is less than about 150 μm.

In certain embodiments, the present invention relates to the aforementioned method, wherein the diameter of said spots is less than about 120 μm and the distance between adjacent spots is less than about 300 μm.

In certain embodiments, the present invention relates to the aforementioned method, wherein said spots comprise said carbohydrate-containing molecule and at least one protein.

In certain embodiments, the present invention relates to the aforementioned method, wherein said array is subdivided into sections using a silicone-rubber gasket.

In certain embodiments, the present invention relates to the aforementioned method, wherein said array comprises a first collection of spots having a first concentration of said carbohydrate-containing molecule, and a second collection of spots having a second concentration of said carbohydrate-containing molecule.

In certain embodiments, the present invention relates to the aforementioned method, wherein said first concentration is not the same as said second concentration.

In certain embodiments, the present invention relates to the aforementioned method, further comprising the step of:
treating said carbohydrate-containing molecules with an enzyme, wherein said enzyme is selected from the group consisting of endoglycosidase, fucosidase, galactosidase, hexosaminidase, hexosidase, mannosidase, neuraminidase, xylosidase, fucosyltransferase, galactosyltransferase, mannosyltransferase, and sialyltransferase.
   Methods for Detection of Substrate Binding on Microarray

Another aspect of the present invention relates to a method to detect the interaction of a carbohydrate with a binding molecule, comprising the steps of:
contacting a binding molecule to an array of carbohydrate-containing molecules comprising a plurality of spots that are less than about 500 μm wide and is within about 900 μm of an adjacent spot to give an analysis sample; and detecting the presence of a complex formed between said carbohydrate-containing molecules and said binding molecule of said analysis sample.

In certain embodiments, the present invention relates to the aforementioned method, wherein said carbohydrate-containing molecule is a monosaccharide, disaccharide, oligosaccharide, polysaccharide, glycoprotein, neoglycopeptide, or glycolipid.

In certain embodiments, the present invention relates to the aforementioned method, wherein said carbohydrate-containing molecule is a monosaccharide, disaccharide, oligosaccharide, or polysaccharide.

In certain embodiments, the present invention relates to the aforementioned method, wherein said carbohydrate-containing molecule is a monosaccharide or oligosaccharide.

In certain embodiments, the present invention relates to the aforementioned method, wherein said carbohydrate-containing molecule is a monasaccharide, trisaccharide, or hexasaccharide.

In certain embodiments, the present invention relates to the aforementioned method, wherein said carbohydrate-containing molecule is a monosaccharide.

In certain embodiments, the present invention relates to the aforementioned method, wherein said carbohydrate-containing molecule is mannose, galactose, lactose, or Man9.

In certain embodiments, the present invention relates to the aforementioned method, wherein said carbohydrate-containing molecule is mannose or galactose.

In certain embodiments, the present invention relates to the aforementioned method, wherein said carbohydrate-containing molecule is a glycoprotein or neoglycopeptide.

In certain embodiments, the present invention relates to the aforementioned method, wherein said carbohydrate-containing molecule is gp120, gp41, gp41-r, invertase, or non-glycosylated g120.

In certain embodiments, the present invention relates to the aforementioned method, wherein said carbohydrate-containing molecule is gp120 or gp41.

In certain embodiments, the present invention relates to the aforementioned method, wherein the diameter of said spots is less than about 300 μm.

In certain embodiments, the present invention relates to the aforementioned method, wherein the diameter of said spots is less than about 200 μm.

In certain embodiments, the present invention relates to the aforementioned method, wherein the diameter of said spots is less than about 120 μm.

In certain embodiments, the present invention relates to the aforementioned method, wherein the diameter of said spots is less than about 80 μm.

In certain embodiments, the present invention relates to the aforementioned method, wherein the distance between adjacent spots is less than about 300 μm.

In certain embodiments, the present invention relates to the aforementioned method, wherein the distance between adjacent spots is less than about 150 μm.

In certain embodiments, the present invention relates to the aforementioned method, wherein the diameter of said spots is less than about 120 μm and the distance between adjacent spots is less than about 300 μm.

In certain embodiments, the present invention relates to the aforementioned method, wherein said spots comprise said carbohydrate-containing molecule and at least one protein.

In certain embodiments, the present invention relates to the aforementioned method, wherein said array comprises a first collection of spots having a first concentration of said carbohydrate-containing molecule, and a second collection of spots having a second concentration of said carbohydrate-containing molecule.

In certain embodiments, the present invention relates to the aforementioned method, wherein said binding molecule is DNA, RNA, protein, lipid, glycoprotein, glycolipid, carbohydrate, or small organic molecule that contains a detectable functional group.

In certain embodiments, the present invention relates to the aforementioned method, wherein said binding molecule is a protein or glycoprotein comprising a detectable functional group.

In certain embodiments, the present invention relates to the aforementioned method, wherein said binding molecule is DNA, RNA, protein, lipid, glycoprotein, glycolipid, carbohydrate, or small organic molecule comprising a detectable functional group.

In certain embodiments, the present invention relates to the aforementioned method, wherein said binding molecule is a protein or glycoprotein comprising a detectable functional group.

In certain embodiments, the present invention relates to the aforementioned method, wherein said binding molecule is DNA, RNA, protein, lipid, glycoprotein, glycolipid, carbohydrate, or small organic molecule comprising a functional group that is detectable by fluorescence spectroscopy.

In certain embodiments, the present invention relates to the aforementioned method, wherein said binding molecule is a protein or glycoprotein comprising a functional group detectable by fluorescence spectroscopy.

In certain embodiments, the present invention relates to the aforementioned method, wherein said binding molecule is BODIPY-labeled cyanoviron-N, FITC-labeled Concanavalin A, Texas Red-labeled Erythrina cristagalli (ECA), DC-SIGN, 2G12-N, CD4, FITC-SAV, or CY3-labeled 2G12.

In certain embodiments, the present invention relates to the aforementioned method, wherein said binding molecule is BODIPY-labeled cyanoviron-N or FITC-labeled Concanavalin A.

In certain embodiments, the present invention relates to the aforementioned method, wherein said binding molecule is BODIPY-labeled cyanoviron-N or FITC-labeled Concanavalin A.

In certain embodiments, the present invention relates to the aforementioned method, wherein said analysis sample is treated with BODIPY-labeled cyanoviron-N, FITC-labeled Concanavalin A, DC-SIGN, 2G12-N, CD4, FITC-SAV, or CY3-labeled 2G12.

Methods for Detection of Substrate Binding on Microsphere Array

Another aspect of the present invention relates to a method of detecting an interaction between a carbohydrate-containing molecule and a binding molecule, comprising the steps of:
contacting a binding molecule to a carbohydrate-containing molecule attached to the surface of a microsphere; and detecting a complex comprising said carbohydrate-containing molecule and said binding molecule.

In certain embodiments, the present invention relates to the aforementioned method, wherein said carbohydrate-containing molecule is a monosaccharide, disaccharide, oligosaccharide, polysaccharide, glycoprotein, neoglycopeptide, or glycolipid.

In certain embodiments, the present invention relates to the aforementioned method, wherein said carbohydrate-containing molecule is a monosaccharide, disaccharide, oligosaccharide, or polysaccharide.

In certain embodiments, the present invention relates to the aforementioned method, wherein said carbohydrate-containing molecule is a monosaccharide or oligosaccharide.

In certain embodiments, the present invention relates to the aforementioned method, wherein said carbohydrate-containing molecule is a monosaccharide.

In certain embodiments, the present invention relates to the aforementioned method, wherein said carbohydrate-containing molecule is mannose or galactose.

In certain embodiments, the present invention relates to the aforementioned method, wherein said carbohydrate-containing molecule is a glycoprotein or neoglycopeptide.

In certain embodiments, the present invention relates to the aforementioned method, wherein said binding molecule is DNA, RNA, protein, lipid, glycoprotein, glycolipid, carbohydrate, or small organic molecule comprising a detectable functional group.

In certain embodiments, the present invention relates to the aforementioned method, wherein said binding molecule is a protein or glycoprotein comprising a detectable functional group.

In certain embodiments, the present invention relates to the aforementioned method, wherein said binding molecule is DNA, RNA, protein, lipid, glycoprotein, glycolipid, carbohydrate, or small organic molecule comprising a detectable functional group.

In certain embodiments, the present invention relates to the aforementioned method, wherein said binding molecule is a protein or glycoprotein comprising a detectable functional group.

In certain embodiments, the present invention relates to the aforementioned method, wherein said binding molecule is DNA, RNA, protein, lipid, glycoprotein, glycolipid, carbohydrate, or small organic molecule comprising a functional group that is detectable by fluorescence spectroscopy.

In certain embodiments, the present invention relates to the aforementioned method, wherein said binding molecule is a protein or glycoprotein comprising a functional group detectable by fluorescence spectroscopy.

In certain embodiments, the present invention relates to the aforementioned method, wherein said binding molecule is BODIPY-labeled cyanoviron-N, Texas Red-labeled Erythrina cristagalli (ECA), FITC-labeled Concanavalin A, DC-SIGN, 2G12-N, CD4, FITC-SAV, or CY3-labeled 2G12.

In certain embodiments, the present invention relates to the aforementioned method, wherein said binding molecule is BODIPY-labeled cyanoviron-N, Texas Red-labeled Erythrina cristagalli (ECA), or FITC-labeled Concanavalin A.

In certain embodiments, the present invention relates to the aforementioned method, wherein said binding molecule is BODIPY-labeled cyanoviron-N.

In certain embodiments, the present invention relates to the aforementioned method, wherein said microsphere consists essentially of glass.

In certain embodiments, the present invention relates to the aforementioned method, wherein said microsphere consists essentially of glass and at least one fluorescent dye.

In certain embodiments, the present invention relates to the aforementioned method, wherein said carbohydrate-containing molecule is attached to said microsphere by a covalent bond.

In certain embodiments, the present invention relates to the aforementioned method, wherein said carbohydrate-containing molecule is attached to said microsphere though a glycosidic linkage.

In certain embodiments, the present invention relates to the aforementioned method, wherein said carbohydrate-containing molecule is attached to said microsphere by a linker.

In certain embodiments, the present invention relates to the aforementioned method, wherein said carbohydrate-containing molecule is attached to said linker by a sulfide bond.

In certain embodiments, the present invention relates to the aforementioned method, wherein said linker is bovine serum albumin.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

Detection of Monsacharides by Concanavalin A and Erythrina Cristagalli

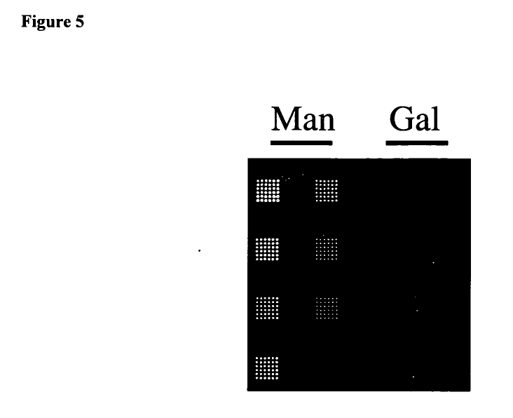

Protein-carbohydrate interactions were examined using the mannose/glucose specific lectin Concanavalin A (ConA). Microarrays were constructed through the maleimide derivatization of BSA coated glass slides to create a thiol-reactive surface (FIG. 1). Thiol derivatized mannose and galactose were printed as 120 μm spots using a microarray printing robot. The remaining maleimide groups were subsequently quenched with a solution of 3-mercaptopropionic acid to render the slides unreactive to cysteine containing proteins. The carbohydrate microarrays were incubated with a solution of FITC-labeled ConA. The arrays were thoroughly rinsed with buffer, dried by centrifugation and scanned with a fluorescence slide scanner. As anticipated, FITC-labeled ConA was observed on the spots corresponding to immobilized mannose, while no fluorescence was associated with the galactose spots (FIG. 5). This result confirms that the microarray platform can be used for the immobilization of carbohydrates while maintaining specificity in carbohydrate-protein interactions. Utilizing proteins conjugated to fluorophores of non-overlapping excitation and-emission spectra enabled facile two-color detection. This strategy can be extended beyond two-color analysis as previously demonstrated. See Bidlingmaier, S.; Synder, M. Chemistry and Biology 2002, 9, 400. It should also be noted that we observed very high signal-to-noise ratios in these experiments, presumably due to minimal nonspecific protein-surface interactions. This is in agreement with the low background levels observed in protein microarrays that also made use of derivatized BSA for the immobilization of small molecules.

Detection of High-Mannose Type Oligiosaccharides by Cyanovirin-N

A series of high-mannose type oligosacoharides was selected, of key importance in the N-linked glycosylation pathway and also found on the glycoproteins of a variety of infectious agents such as human immunodeficiency virus, influenza virus and trypanosomes. See S. Magez et al. J. Biol. Chem. 2001, 276, 33458. We chose the protein cyanovirin-N (CVN) as a model case for the study of protein-oligosaccharide binding events. See B. O'Keefe et al. Mol. Pharmacol. 2000, 58, 982. Isolated from the blue-green-algae Nostoc elliposporum, CVN was found to bind the high-mannose oligosaccharides of gp120, thereby inhibiting HIV's ability to infect T cells. Natural and recombinant forms of CVN have been shown to irreversibly inactivate a wide variety of HIV strains while exhibiting minimal toxicity to host cells. See M. Boyd et al. Antimicrob. Agents Chemother. 1997,41, 1521. The ability of CVN to bind high-mannose oligosaccharides rendered it an ideal test case for a carbohydrate array containing synthetic oligosaccharides of different lengths and complexity.

Using the aforementioned thiol terminated linker, a series of high-mannose oligosaccharides were prepared (Scheme 6). Arrays were printed on a BSA coated slide, as above, varying the concentration of printed oligosaccharide, and blocked with 3-mercaptoproprionic acid. After incubation with BODIPY-labeled CVN, fluorescence was detected at spots corresponding to the, immobilized linear trimannoside 3, hexamannoside 5 and nonamannoside while the branched trimannoside 4, mannose 1, and galactose 2 showed no binding activity.

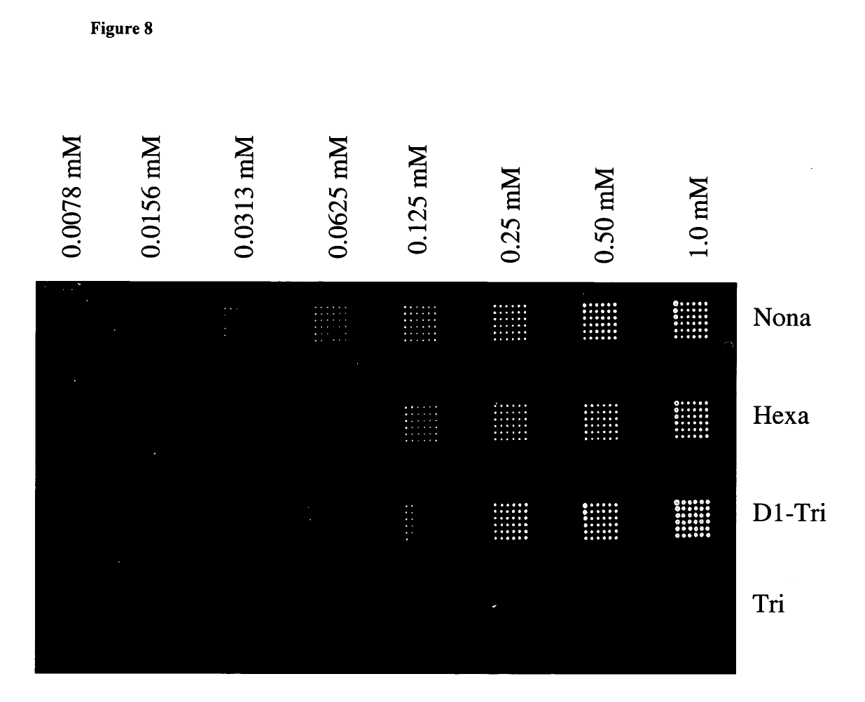

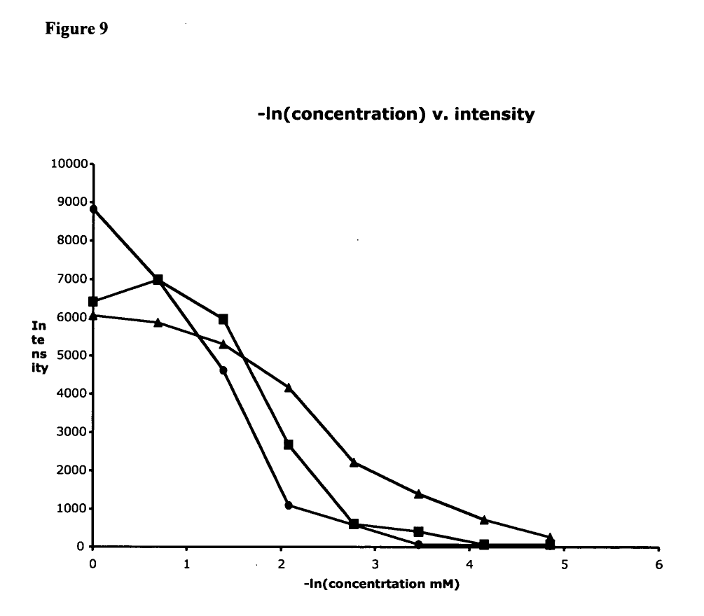

It was of interest to establish whether the microarray platform may serve to probe relative affinities of CVN for the different high-mannose structures. A microarray containing the four oligosaccharide structures was prepared with carbohydrate concentrations ranging from 0.008 mM to 2 mM. This dilution series microarray exhibited a CVN binding pattern that reflects the relative affinities of CVN for the various structures (FIG. 8) and is in full agreement with a recent isothermocalorimetry and NMR study that determined binding of CVN to nonamannoside (K_{d}=0.27 μM), hexamannoside (K_{d}=2.61 NLM), and linear trimannoside (K_{d}=3.48 μM). Here we see that nonamannoside, for which CVN bears the highest affinity, showed discernable fluorescence at concentrations where the signals of the other structures were unobservable. In FIG. 9, the plotted spot intensities from the dilution series (normalized to background) reveal that at higher, concentrations (1 mM-0.5 mM) both the linear trimannoside and the hexamannoside have higher fluorescence intensities than the nonamannoside. At lower dilutions, however, this trend is reversed. This discrepancy from the isocalorimetry data can be explained by the density of covalently immobilized saccharide at saturating concentrations. We hypothesize that the size of the nonasaccharide, compared to the other structures immobilized, might sterically occlude reactive sites leading to decreased amounts of immobilized structure. As previously observed, two distinct CVN binding domains can each interact with a single α1,2-linked linear trimannoside. We reason that a higher surface concentrations of 3 permits this multivalent interaction to take place.

Experimental Procedures:

Carbohydrate synthesis: Thiol-terminated ethylene glycol derivitized saccharides were prepared as described in the literature. See P. Seeberger et al. Eur. J. Org. Chem. 2002, 5, 826. In the syntheses, 2-[2-(2-Benzylsylfanylethoxy)-ethoxy]-ethanol was substituted for pentenyl alcohol. This substitution affords an ethylene glycol modified thiol handle for covalent immobilization of the structures to a maleimide modified surface.

Functionalization of slides: SuperAldehyde slides (TeleChem International) were immersed in 50 mL phosphate buffered saline (PBS) containing 1% bovine serum albumin (BSA; w/v) and incubated overnight at room temperature. The slides were rinsed twice with distilled H₂O (100 mL), twice with 95% ethanol (50 mL) and then dried under a stream of dry Ar. Thereafter the slides were immersed in 45 mL of anhydrous DMF (Aldrich) containing 65 mg succinimidyl-4-(Nmaleimidomethyl)cyclohexane-1-carboxyiate. (Pierce Chemical) and 100 mM N,N-Diisopropylethylamine (Aldrich). The slides were incubated in this solution for 24 hours at room temperature, washed 4 times with 95% ethanol (50 mL) and then stored in a vacuum dessicator until used for microarray fabrication.

Microarray fabrication: Thiol functionalized carbohydrates were incubated 1 hr at room temperature with 1 equivalent tris-(carboxyethyl)phosphine hydrochloride (TCEP) in 1×PBS, and printed on the maleimide derivitized glass slides using a MicroGrid TAS array printer. Prints were performed at 30% humidity using either a 16-pin or 32-pin format, with a spot size of 120 μm and a distance of 300 μm between the centers of adjacent spots. Thereafter, the slides were stored in a humid chamber at room temperature for 12 hours, washed 2 times with distilled H₂O, and then incubated for 1 hour in 1 mM 3-mercaptopropionic acid in PBS (50 mL) to quench all remaining maleimide groups. The slides were washed three times with distilled H₂O (50 mL), two times with 95% ethanol (50 mL) and then stored in a vacuum dessicator until used for binding experiments.

Detection of protein-carbohydrate interactions: In experiments involving FITC labeled ConA (Sigma), the lectin was used at 25 μg mL⁻¹ in 10 mM HEPES-BSA buffer (pH 7.5, 1 mM CaCl₂, 1 mM MnCl₂, 100 mM NaCl, 1% (w/v) BSA). In experiments involving BODIPY-labeled CVN, the CVN was used at 25 μg mL-1 in 10 mM PBS containing 1% BSA. For all incubations, 0.55 mL of protein solution was applied to the slide using a PC500 CoverWell incubation chamber (Grace BioLabs). Following a 1 hour incubation at room temperature, the slides were washed three times with 50 mL of the same buffer used in the incubation, twice with 50 mL distilled H₂O and then centrifuged at 200 g for 5 minutes to ensure complete dryness. To visualize fluorescence, the slides were scanned using an ArrayWoRx fluorescence slide scanner (Applied Precision).

### Example 2

Immobilization and Binding Analysis of Proteins, Neoglycoproteins, and Glycoproteins

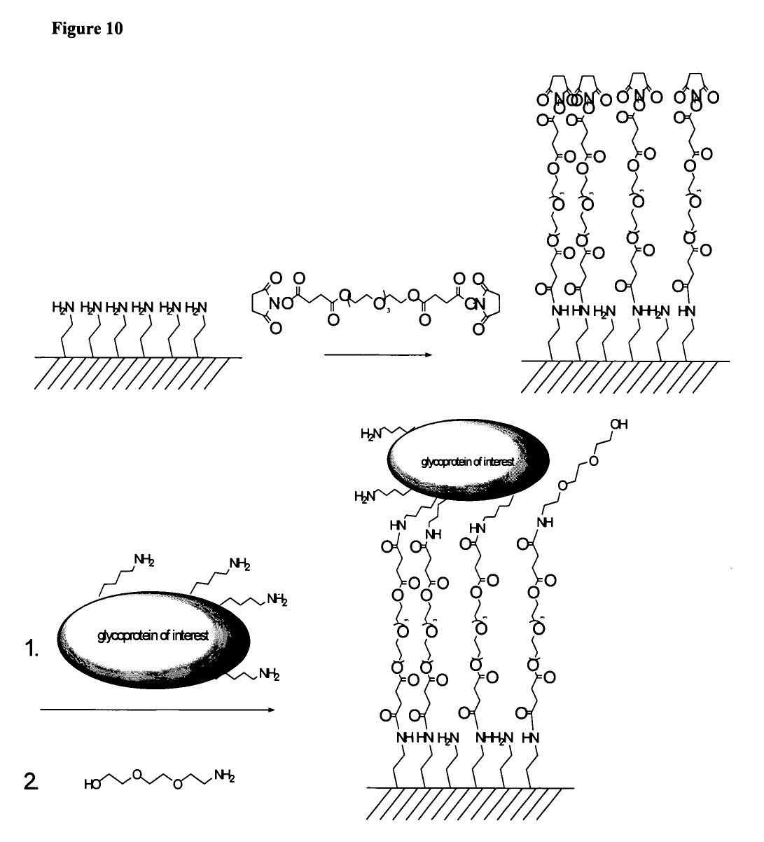

Functionalization of slides: Corning GAPS II amino propyl silane treated slides were immersed in 50 mLs anhydrous DMF containing 100 mM N,N-Diisopropylethylamine base and 10 mg bis-succinimidyl ester tetraethylene glycol (FIG. 10) and incubated overnight at room temperature. The slides were rinsed three times with 95% ethanol (50 mL) and then dried under a stream of dry Ar. Slides were then stored in a vacuum dessicator until used for microarray fabrication.

Microarray fabrication: Proteins, neoglycoproteins, and glycoproteins were printed at high density on functionalized glass slides using a MicroGrid TAS array printer. Prints were performed at 30% humidity using either a 16-pin or 32-pin format, with a spot size of 120 μm and a distance of 300 μm between the centers of adjacent spots. Thereafter the slides were stored in a humid chamber at room temperature for 12 hours, washed 2 times with distilled H₂O, and then incubated for 1 hour in 1 mM 2-(2-(2-aminoethoxy)ethoxy)ethanol in PBS (50 mL) to quench all remaining succinimidyl ester groups. The slides were washed three times with distilled H₂O (50 mL), two times with 95% ethanol (50 mL) and then stored in a vacuum dessicator until used for binding experiments.

Binding Analysis: Binding experiments were carried out based on the procedures described in Example 1. Fluorescence spectroscopy was used to visualize the results of the binding experiments.

### Example 3

Carbohydrate Binding Experiments Using Fiber Optic Microsphere Arrays

To demonstrate the utility of such arrays for studying protein-carbohydrate interactions, we examined two model systems, the mannose binding lectin Concanavalin A (ConA), and cyanovirin N (CVN), a novel HIV-inactivating 11 kDa protein derived from the cyanobacterium Nostoc ellipsosporum with demonstrated specificity for high-mannose oligosaccharides M. Boyd et al. Antimicrobial Agents and Chemotherapy 1997, 41, 1521.

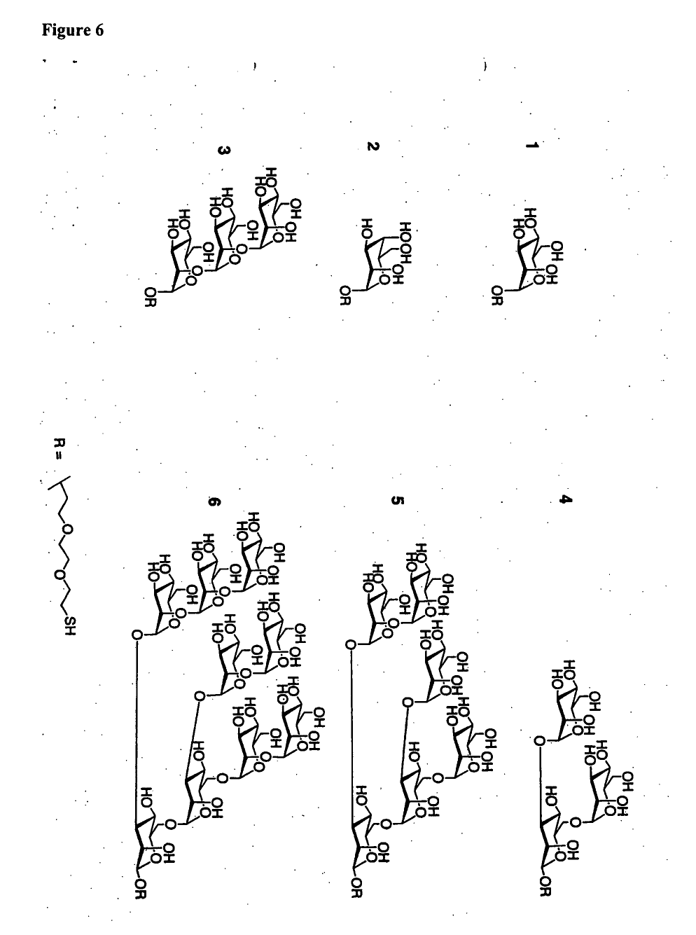

Mannose 1 and galactose 2 monosaccharides-were prepared with an ethylenedioxy thiol-terminated linker at the anomeric center (FIG. 6). Each monosaccharide was coupled to commercially available maleimide-activated bovine serum albumin (BSA). The prepared neoglycoproteins were then attached to encoded microspheres with a water soluble carbodiimide and used to form a randomly ordered fiber optic microsphere array. ConA binding was detected by incubating the fiber optic array in a solution of tetramethyrhodamine labeled-ConA at 50 μg mL⁻¹ for five minutes. At five minutes, the lectin solution was removed, replaced with a fresh buffer solution and the fluorescence signal at the rhodamine wavelength was measured. Only those beads bearing 1 were bound by labeled ConA. The signal from empty wells and beads bearing immobilized 2 reveals that no ConA was observed in the empty wells or associated with 2 modified beads. Thus the intrinsic non-specific binding levels of fiber optic arrays in this assay were very low and the specificity of carbohydrate-protein interactions was clearly observed.

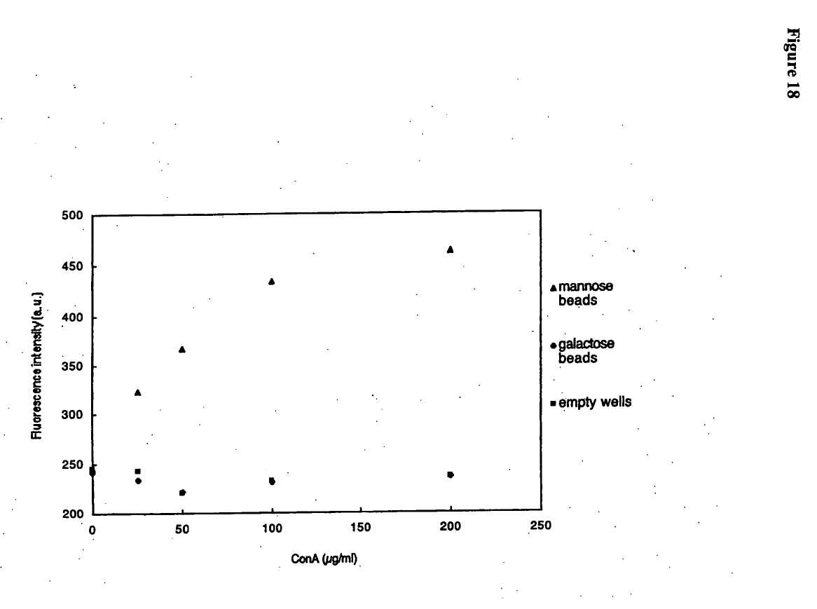

To determine what concentrations of labeled ConA were required in solution to observe signals easily discriminated over background, prepared fiber optic arrays were incubated with a dilution series of labeled ConA ranging from 0 to 400 μg mL⁻¹. FIG. 18 shows the results of these tests and it is clear that ConA concentrations as low as 25 μg mL⁻¹ yielded fluorescence signals easily discernible over background. It is also noteworthy that even at very high lectin concentrations no increase in fluorescence is observed associated with the beads presenting 2.

Having detected protein-carbohydrate interactions with a simple lectin-monosaccharide system, we wished to establish if the fiber optic arrays could also be applied to more complex oligosaccharide structures. For this test we chose cyanovirin N (CV-N) and its interactions with high-mannose oligosaccharides.

Five synthetic carbohydrates (1,3-6) were immobilized on microspheres carrying a unique internal code for each structure. A small aliquot of each of the five dispersions was mixed together and used to microsphere array.

CV-N oligosaccharide binding was assayed by incubating the formed array in a solution of BODIPY-labeled CV-N at 50 μg mL⁻¹ for five minutes. At five minutes the CV-N solution was removed and replaced with a fresh buffer solution. BODIPY excitation wavelength (488 nm) was passed through the optical fiber and BODIPY emission wavelengths were collected on the CCD camera. Three of the five structures (3,5,6) present were bound by CV-N in accordance with isothermal microcalorimetry studies P. H. Seeberger et al. Chemistry and Biology 2002, 9, 1109. Beads that were not bound by CV-N did not show any fluorescence signals over background levels.

In summary, we have demonstrated that randomly ordered fiber optic microsphere arrays bearing immobilized synthetic oligosaccharides can be used to evaluate protein-carbohydrate interactions. The system we described here allowed for simultaneous evaluation of five distinct structures against a carbohydrate binding protein of interest in a rapid fashion and with unambiguous results. As the specificity of the protein-carbohydrate interactions observed with the microarray mirror those observed in solution studies, we anticipate that these arrays will be useful tools for the evaluation of protein-carbohydrate interactions.

Experimental Procedures

Neoglycoprotein preparation: Maleimide-activated bovine serum albumin (BSA) and Tris(2-carboxyethyl)phosphine hydrochloride (TCEP) were purchased from Pierce Chemical. 50 μg (152 nmol) of 1 were incubated with 1 equivalent TCEP in 10 mM HEPES buffer (pH 7.5) at room temperature for 1 hour with constant mixing. This solution was then added to 100 μg maleimide-modified BSA in 100 μL of the same buffer. The solution was incubated overnight at room temperature with constant mixing. Without further purification the microspheres prepared below were added to this neoglycoprotein solution. This coupling chemistry was used for all structures used in this study.

Microsphere preparation: 4.4 μg QuantumPlex internally-encoded microspheres were purchased from Bangs Laboratories. 1-Ethyl-3-(3-dimethylaminopropyl)carbodiimide hydrochloride (EDC) and Tween-20 were purchased from Aldrich. 100 μL of stock beads were washed and conjugated to neoglycoproteins according to the manufacturer's instructions. Conjugation reactions were performed in 10 mM HEPES buffer (pH 7.5) Microspheres were purified away from excess neoglycoproteins by centrifugation and the final microsphere pellet was redispersed in 10 mM HEPES. The dispersions were stored at 4° C. until further use.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. An array, comprising a plurality of spots on a solid support, wherein each spot independently comprises a substrate attached to said solid support, wherein each substrate attached to said solid support is independently a carbohydrate-containing molecule.

2. The array of claim 1, wherein said solid support is glass, gold-coated glass, polymer, or metal surface.

3. The array of claim 1, wherein said solid support is glass or gold-coated glass.

4. The array of claim 1, wherein said solid support is glass.

5. The array of claim 1, wherein said carbohydrate-containing molecule is a monosaccharide, disaccharide, oligosaccharide, polysaccharide, glycoprotein, neoglycopeptide, or glycolipid.

6. The array of claim 1, wherein said carbohydrate-containing molecule is a monosaccharide, disaccharide, oligosaccharide, or polysaccharide.

7. The array of claim 1, wherein said carbohydrate-containing molecule is a monosaccharide or oligosaccharide.

8. The array of claim 1, wherein said carbohydrate-containing molecule is a monasaccharide, trisaccharide, or hexasaccharide.

9. The array of claim 1, wherein said carbohydrate-containing molecule is a monosaccharide.

10. The array of claim 1, wherein said carbohydrate-containing molecule is mannose, galactose, lactose, or Man9.

11. The array of claim 1, wherein said carbohydrate-containing molecule is mannose or galactose.

12. The array of claim 1, wherein said carbohydrate-containing molecule is mannose.

13. The array of claim 1, wherein said carbohydrate-containing molecule is a glycoprotein or neoglycopeptide.

14. The array of claim 1, wherein said carbohydrate-containing molecule is gp120, gp41, gp41-r, invertase, or non-glycosylated g120.

15. The array of claim 1, wherein said carbohydrate-containing molecule is gp120 or gp41.

16. The array of claim 1, wherein said carbohydrate-containing molecule is attached to said solid support by a non-covalent interaction.

17. The array of claim 1, wherein said carbohydrate-containing molecule is attached to said solid support by a covalent bond.

18. The array of claim 1, wherein said carbohydrate-containing molecule is attached to said solid support by a linker.

19. The array of claim 18, wherein said carbohydrate-containing molecule is attached to said linker by a sulfide bond.

20. The array of claim 18, wherein said carbohydrate-containing molecule is attached to said linker though a glycosidic linkage.

21. The array of claim 18, wherein said linker is bovine serum albumin.

22. The array of claim 1, wherein the diameter of said spots is less than about 300 μm.

23. The array of claim 1, wherein the diameter of said spots is less than about 200 μm.

24. The array of claim 1, wherein the diameter of said spots is less than about 120 μm.

25. The array of claim 1, wherein the diameter of said spots is less than about 80 μm.

26. The array of claim 1, wherein the distance between adjacent spots is less than about 900 μm.

27. The array of claim 1, wherein the distance between adjacent spots is less than about 500 μm.

28. The array of claim 1, wherein the distance between adjacent spots is less than about 300 μm.

29. The array of claim 1, wherein the distance between adjacent spots is less than about 150 μm.

30. The array of claim 1, wherein the diameter of said spots is less than about 120 μm and the distance between adjacent spots is less than about 300 μm.

31. The array of claim 1, wherein said spots comprise said carbohydrate-containing molecule and at least one protein.

32. The array of claim 1, wherein said array is subdivided into sections using a silicone-rubber gasket.

33. The array of claim 1, wherein said array comprises a first collection of spots having a first concentration of said carbohydrate-containing molecule, and a second collection of spots having a second concentration of said carbohydrate-containing molecule.

34. A method of preparing an array of carbohydrate-containing molecules, comprising the step of:
applying a carbohydrate-containing molecule to a solid support to form a spot that has a diameter less than about 500 μm.

35. The method of claim 34, wherein said solid support is glass, gold-coated glass, polymer, or metal surface.

36. The method of claim 34, wherein said solid support is glass or gold-coated glass.

37. The method of claim 34, wherein said solid support is glass.

38. The method of claim 34, wherein said carbohydrate-containing molecule is a monosaccharide, disaccharide, oligosaccharide, polysaccharide, glycoprotein, neoglycopeptide, or glycolipid.

39. The method of claim 34, wherein said carbohydrate-containing molecule is a monosaccharide; disaccharide, oligosaccharide, or polysaccharide.

40. The method of claim 34, wherein said carbohydrate-containing molecule is a monosaccharide or oligosaccharide.

41. The method of claim 34, wherein said carbohydrate-containing molecule is a monasaccharide, trisaccharide, or hexasaccharide.

42. The method of claim 34, wherein said carbohydrate-containing molecule is a monosaccharide.

43. The method of claim 34, wherein said carbohydrate-containing molecule is mannose, galactose, lactose, or Man9.

44. The method of claim 34, wherein said carbohydrate-containing molecule is mannose or galactose.

45. The method of claim 34, wherein said carbohydrate-containing molecule is mannose.

46. The method of claim 34, wherein said carbohydrate-containing molecule is a glycoprotein or neoglycopeptide.

47. The method of claim 34, wherein said carbohydrate-containing molecule is gp120, gp41, gp41-r, invertase, or non-glycosylated g120.

48. The method of claim 34, wherein said carbohydrate-containing molecule is gp120 or gp41.

49. The method of claim 34, wherein said carbohydrate-containing molecule is attached to said solid support by a noncovalent interaction.

50. The method of claim 34, wherein said carbohydrate-containing molecule is attached to said solid support by a covalent bond.

51. The method of claim 34, wherein said carbohydrate-containing molecule is attached to said solid support by a linker.

52. The method of claim 51, wherein said carbohydrate-containing molecule is attached to said linker by a sulfide bond.

53. The method of claim 51, wherein said carbohydrate-containing molecule is attached to said linker though a glycosidic linkage.

54. The method of claim 51, wherein said linker is bovine serum albumin.

55. The method of claim 34, wherein the diameter of said spots is less than about 300 μm.

56. The method of claim 34, wherein the diameter of said spots is less than about 200 μm.

57. The method of claim 34, wherein the diameter of said spots is less than about 120 μm.

58. The method of claim 34, wherein the diameter of said spots is less than about 80 μm.

59. The method of claim 34, wherein the distance between adjacent said spots is less than about 300 μm.

60. The method of claim 34, wherein the distance between adjacent said spots is less than about 150 μm.

61. The method of claim 34, wherein the diameter of said spots is less than about 120 μm and the distance between adjacent spots is less than about 300 μm.

62. The method of claim 34, wherein said spots comprise said carbohydrate-containing molecule and at least one protein.

63. The method of claim 34, wherein said array is subdivided into sections using a silicone-rubber gasket.

64. The method of claim 34, wherein said array comprises a first collection of spots having a first concentration of said carbohydrate-containing molecule, and a second collection of spots having a second concentration of said carbohydrate-containing molecule.

65. The method of claim 64, wherein said first concentration is not the same as said second concentration.

66. The method of claim 34, further comprising the step of:
treating said carbohydrate-containing molecules with an enzyme, wherein said enzyme is selected from the group consisting of endoglycosidase, fucosidase, galactosidase, hexosaminidase, hexosidase, mannosidase, neuraminidase, xylosidase, fucosyltransferase, galactosyltransferase, mannosyltransferase, and sialyltransferase.

67. A method to detect the interaction of a carbohydrate with a binding molecule, comprising the steps of:
contacting a binding molecule to an array of carbohydrate-containing molecules comprising a plurality of spots that are less than about 500 μm wide and is within about 900 μm of an adjacent spot to give an analysis sample; and detecting the presence of a complex formed between said carbohydrate-containing molecules and said binding molecule of said analysis sample.

68. The method of claim 67, wherein said carbohydrate-containing molecule is a monosaccharide, disaccharide, oligosaccharide, polysaccharide, glycoprotein, neoglycopeptide, or glycolipid.

69. The method of claim 67, wherein said carbohydrate-containing molecule is a monosaccharide, disaccharide, oligosaccharide, or polysaccharide.

70. The method of claim 67, wherein said carbohydrate-containing molecule is a monosaccharide or oligosaccharide.

71. The method of claim 67, wherein said carbohydrate-containing molecule is a monasaccharide, trisaccharide, or hexasaccharide.

72. The method of claim 67, wherein said carbohydrate-containing molecule is a monosaccharide.

73. The method of claim 67, wherein said carbohydrate-containing molecule is mannose, galactose, lactose, or Man9.

74. The method of claim 67, wherein said carbohydrate-containing molecule is mannose or galactose.

75. The method of claim 67, wherein said carbohydrate-containing molecule is a glycoprotein or neoglycopeptide.

76. The method of claim 67, wherein said carbohydrate-containing molecule is gp120, gp41, gp41-r, invertase, or non-glycosylated g120.

77. The method of claim 67, wherein said carbohydrate-containing molecule is gp120 or gp41.

78. The method of claim 67, wherein the diameter of said spots is less than about 300 μm.

79. The method of claim 67, wherein the diameter of said spots is less than about 200 μm.

80. The method of claim 67, wherein the diameter of said spots is less than about 120 μm.

81. The method of claim 67, wherein the diameter of said spots is less than about 80 μm.

82. The method of claim 67, wherein the distance between adjacent spots is less than about 300 μm.

83. The method of claim 67, wherein the distance between adjacent spots is less than about 150 μm.

84. The method of claim 67, wherein the diameter of said spots is less than about 120 μm and the distance between adjacent spots is less than about 300 μm.

85. The method of claim 67, wherein said spots comprise said carbohydrate-containing molecule and at least one protein.

86. The method of claim 67, wherein said array comprises a first collection of spots having a first concentration of said carbohydrate-containing molecule, and a second collection of spots having a second concentration of said carbohydrate-containing molecule.

87. The method of claim 67, wherein said binding molecule is DNA, RNA, protein, lipid, glycoprotein, glycolipid, carbohydrate, or small organic molecule that contains a detectable functional group.

88. The method of claim 67, wherein said binding molecule is a protein or glycoprotein comprising a detectable functional group.

89. The method of claim 67, wherein said binding molecule is DNA, RNA, protein, lipid, glycoprotein, glycolipid, carbohydrate, or small organic molecule comprising a detectable functional group.

90. The method of claim 67, wherein said binding molecule is a protein or glycoprotein comprising a detectable functional group.

91. The method of claim 67, wherein said binding molecule is DNA, RNA, protein, lipid, glycoprotein, glycolipid, carbohydrate, or small organic molecule comprising a functional group that is detectable by fluorescence spectroscopy.

92. The method of claim 67, wherein said binding molecule is a protein or glycoprotein comprising a functional group detectable by fluorescence spectroscopy.

93. The method of claim 67, wherein said binding molecule is BODIPY-labeled cyanoviron-N, FITC-labeled Concanavalin A, Texas Red-labeled Erythrina cristagalli (ECA), DC-SIGN, 2G12-N, CD4, FITC-SAV, or CY3-labeled 2G12.

94. The method of claim 67, wherein said binding molecule is BODIPY-labeled cyanoviron-N or FITC-labeled Concanavalin A.

95. The method of claim 67, wherein said binding molecule is BODIPY-labeled cyanoviron-N or FITC-labeled Concanavalin A.

96. The method of claim 67, wherein said analysis sample is treated with BODIPY-labeled cyanoviron-N, FITC-labeled Concanavalin A, DC-SIGN, 2G12-N, CD4, FITC-SAV, or CY3-labeled 2G12.

97. A method of detecting an interaction between a carbohydrate-containing molecule and a binding molecule, comprising the steps of:
contacting a binding molecule to a carbohydrate-containing molecule attached to the surface of a microsphere; and detecting a complex comprising said carbohydrate-containing molecule and said binding molecule.

98. The method of claim 97, wherein said carbohydrate-containing molecule is a monosaccharide, disaccharide, oligosaccharide, polysaccharide, glycoprotein, neoglycopeptide, or glycolipid.

99. The method of claim 97, wherein said carbohydrate-containing molecule is a monosaccharide, disaccharide, oligosaccharide, or polysaccharide.

100. The method of claim 97, wherein said carbohydrate-containing molecule is a monosaccharide or oligosaccharide.

101. The method of claim 97, wherein said carbohydrate-containing molecule is a monosaccharide.

102. The method of claim 97, wherein said carbohydrate-containing molecule is mannose or galactose.

103. The method of claim 97, wherein said carbohydrate-containing molecule is a glycoprotein or neoglycopeptide.

104. The method of claim 97, wherein said binding molecule is DNA, RNA, protein, lipid, glycoprotein, glycolipid, carbohydrate, or small organic molecule comprising a detectable functional group.

105. The method of claim 97, wherein said binding molecule is a protein or glycoprotein comprising a detectable functional group.

106. The method of claim 97, wherein said binding molecule is DNA, RNA, protein, lipid, glycoprotein, glycolipid, carbohydrate, or small organic molecule comprising a detectable functional group.

107. The method of claim 97, wherein said binding molecule is a protein or glycoprotein comprising a detectable functional group.

108. The method of claim 97, wherein said binding molecule is DNA, RNA, protein, lipid, glycoprotein, glycolipid, carbohydrate, or small organic molecule comprising a functional group that is detectable by fluorescence spectroscopy.

109. The method of claim 97, wherein said binding molecule is a protein or glycoprotein comprising a functional group detectable by fluorescence spectroscopy.

110. The method of claim 97, wherein said binding molecule is BODIPY-labeled cyanoviron-N, Texas Red-labeled Erythrina cristagalli (ECA), FITC-labeled Concanavalin A, DC-SIGN, 2G12-N, CD4, FITC-SAV, or CY3-labeled 2G12.

111. The method of claim 97, wherein said binding molecule is BODIPY-labeled cyanoviron-N, Texas Red-labeled Erythrina cristagalli (ECA), or FITC-labeled Concanavalin A.

112. The method of claim 97, wherein said binding molecule is BODIPY-labeled cyanoviron-N.

113. The method of claim 97, wherein said microsphere consists essentially of glass.

114. The method of claim 97, wherein said microsphere consists essentially of glass and at least one fluorescent dye.

115. The method of claim 97, wherein said carbohydrate-containing molecule is attached to said microsphere by a covalent bond.

116. The method of claim 97, wherein said carbohydrate-containing molecule is attached to said microsphere though a glycosidic linkage.

117. The method of claim 97, wherein said carbohydrate-containing molecule is attached to said microsphere by a linker.

118. The method of claim 117, wherein said carbohydrate-containing molecule is attached to said linker by a sulfide bond.

119. The method of claim 117, wherein said linker is bovine serum albumin.
